(19) United States
(12) Patent Application Publication
(10) Pub. No.: US 2026/0015434 A1
(43) Pub. Date: Jan. 15, 2026

(54) ANTIBODY MATERIALS AND METHODS TARGETING MICROBIAL POLYSACCHARIDES

(71) Applicant: The United States of America, as represented by the Secretary, Department of Health and Human Servic, Bethesda, MD (US)

(72) Inventors: Jeffrey Charles Gildersleeve, Frederick, MD (US); Joel Sebastian Temme, Frederick, MD (US)

(73) Assignee: The United States of America, as represented by the Secretary, Department of Health and Human Servic, Bethesda, MD (US)

(21) Appl. No.: 18/846,077

(22) PCT Filed: Mar. 9, 2023
(86) PCT No.: PCT/US2023/064047

§ 371 Date: Sep. 11, 2024

Related U.S. Application Data
(60) Provisional application No. 63/319,090, filed on Mar. 11, 2022.

(51) Int. Cl. C07K 16/44; A61K 47/68; A61P 31/04; G01N 33/569

(52) U.S. Cl. CPC C07K 16/44; A61K 47/6879; A61P 31/04; G01N 33/56911; C07K 2317/14; C07K 2317/24; C07K 2317/31; C07K 2317/52; C07K 2317/565; C07K 2317/73; C07K 2317/76; C07K 2317/92; G01N 2400/00

(57) ABSTRACT

Antibodies and antigen-binding fragments thereof are provided that bind beta-1,6-poly-N-acetyl glucosamine (PNAG) that has been deacetylated in whole or part (dPNAG). Compositions and kits comprising these antibodies and antigen-binding fragment thereof are also provided. Such compositions can include, for example, diagnostic and therapeutic compositions. Methods of using these antibodies and antigen-binding fragments thereof are further provided. Such methods can include, for example, methods for preventing, diagnosing, and treating bacterial infections and associated biofilms characterized by PNAG and dPNAG.

### CROSS-REFERENCE TO RELATED APPLICATIONS

This is the U.S. National Stage of International Application No. PCT/US2023/064047, filed on Mar. 9, 2023, which was published in English under PCT Article 21 (2), which in turn claims the benefit of priority of U.S. Provisional Patent Application No. 63/319,090, filed on Mar. 11, 2022. The provisional application is incorporated by reference herein in its entirety.

### REFERENCE TO SEQUENCE LISTING

Pursuant to the EFS-Web legal framework and 37 C.F.R. § 1.821-825 (see M.P.E.P. § 2442.03 (a)), a Sequence Listing in the form of an ST.26-compliant XML file (entitled “4239-112224-03_sequence_listing.XML” created on Mar. 12, 2025, and is 81,920 bytes in size) is submitted concurrently with the instant application, and the entire contents of the Sequence Listing are incorporated herein by reference.

### BACKGROUND

Poly-β-1-6-N-acetyl glucosamine (beta-1,6-poly-N-acetyl glucosamine) (PNAG) is an exopolysaccharide produced by a broad spectrum of Gram positive and Gram negative bacteria and has been suggested to be produced by a more diverse assortment of microorganisms, including fungi and protozoa. PNAG is a required structural component and important virulence factor for many biofilm-forming pathogenic and opportunistic commensal bacteria, including Gram-positive bacterial species Staphylococcus epidermidis and Staphylococcus aureus. Primarily infecting immunocompromised patients, S. epidermidis has been isolated from nosocomial infections, including urinary tract infections, infective endocarditis, sepsis, and medical device infections. S. aureus and methicillin resistant S. aureus (MRSA) infections are responsible for 120,000 cases of septicemia and 20,000 deaths in the U.S. per year. PNAG producing microorganisms, including Escherichia coli 0157, Candida albicans, Aspergillus species, Neisseria gonorrhea, and Streptococcus species, are responsible for a significant number of community and hospital acquired infections. Traditional treatments of these pathogens have led to the emergence of antibiotic resistant strains, necessitating the development of novel antibiotic therapeutics. Unlike many surface-associated capsular polysaccharides or bacterial antigens, which are often species-specific antigens, PNAG is a broadly conserved surface polysaccharide that offers a promising target for pan-pathogen therapeutic and vaccine development.

Given the clinical significance of PNAG-producing pathogens, a great deal of effort has been directed at understanding the biosynthesis, structure and function, and immune response to this broadly expressed biopolymer. Post-biosynthetic chemical modifications to exopolysaccharides is a common occurrence in bacterial species. PNAG is biosynthesized intracellularly as a homopolymer of β-1-6-N-acetyl glucosamines. However, partial deacetylation of PNAG (>20%) is required for proper function, surface attachment, and biofilm formation. In Gram negative species E. coli and Bordetella bronchiseptica, the polymer is partially deacetylated in the periplasm by deacetylase enzymes polyglucosamine subunit B (PgaB) and poly-β-1,6-N-acetyl-d-glucosamine deacetylase (BpsB), respectively. It has been demonstrated that deletion of PgaB and the resultant loss of deacetylase activity in E. coli prevents PNAG export. Deletion of the BpsB gene in B. bronchiseptica results in an immature biofilm with altered phenotypic characteristics. In Gram-positive species, deacetylase modification of PNAG is also required for the development of biofilms. In S. epidermidis, PNAG (formerly polysaccharide intercellular adhesin, PIA) is modified by the surface associated deacetylase Intercellular adhesion protein B (IcaB). Multiple studies confirm that deacetylated PNAG (dPNAG) is required for S. epidermidis biofilm formation. Similarly, IcaB S. aureus knockouts result in biofilm-deficient and poorly virulent strains. These and other studies provide ample evidence that deacetylase activity produces a positively charged dPNAG polymer that is both structurally and functionally different than its unaltered precursor, PNAG. There is also a significant amount of evidence that PNAG and dPNAG are differentially recognized by the immune system.

Several lines of evidence have demonstrated that dPNAG is the preferred therapeutic target. Human serum is rich in natural antibodies that bind to fully N-acetylated PNAG. However, these antibodies fail to provide adequate protection against pathogenic or opportunistic pathogens. The immune response of native PNAG (>90% N-acetylated PNAG) has been compared to the immune response of chemically deacetylated PNAG (˜15% N-acetylated PNAG) and found dPNAG to be far superior to PNAG in eliciting a neutralizing and protective antibody response. Multiple synthetic dPNAG vaccine platforms have been developed and initial results support dPNAG to be a promising immunogenic target for a protective immune response. B cells isolated from recovered S. aureus infected human subjects have revealed a monoclonal antibody (mAb), F598, that binds to PNAG and chemically deacetylated PNAG. F598 is effective at engaging the complement system and eliciting opsonic killing (OPK). F598 has been shown in vitro to bind to a broad range of pathogenic microorganisms and has demonstrated efficacy in lethal microbial challenge in multiple animal models, advancing to Phase II clinical trials as a promising broad-spectrum antimicrobial biotherapeutic agent. The promising results of dPNAG vaccines and the impressive characteristics of F598 support that dPNAG recognition is critical for OPK activity and is a promising target for vaccine and monoclonal antibody development.

### BRIEF SUMMARY

Antibodies and antigen-binding fragments thereof are provided that bind beta-1,6-poly-N-acetyl glucosamine (PNAG) that has been deacetylated in whole or part (dPNAG). That is, these antibodies and fragments thereof can bind saccharides comprising beta-1,6,-linked glucosamine residues. These antibodies and antibody binding fragments include, for example, those that can bind 1,6-polyglucosamine, or 1,6-poly-N-acetyl glucosamine, or both. Compositions and kits comprising these antibodies and antigen-binding fragment thereof are also provided. Such compositions can include, for example, diagnostic and therapeutic compositions. Methods of using these antibodies and antigen-binding fragments thereof are further provided. Such methods can include, for example, methods for preventing, diagnosing, and treating bacterial infections and associated biofilms characterized by PNAG and dPNAG.

An antibody or antigen-binding fragment thereof is provided that comprises a heavy chain variable region and a light chain variable region, the heavy chain variable region having an amino acid sequence comprising SEQ ID NO: 3, and the light chain variable region having an amino acid sequence comprising SEQ ID NOS: 12 or 24. An antibody comprising a heavy chain variable region and a light chain variable region is provided; wherein the heavy chain variable region comprises an amino acid sequence selected from SEQ ID NO: 3, 31, 34, 36-39, 41, and 42; and wherein the light chain variable region comprises an amino acid sequence selected from SEQ ID NOS: 12, 24, 40, and 43. An antibody or antigen-binding fragment thereof comprising a heavy chain variable region and a light chain variable region is provided, wherein the heavy chain variable region comprises an amino acid sequence selected from SEQ ID NOS: 3, 31, 34, 36-39, 41, and 42 and wherein the light chain variable region having an amino acid sequence comprising SEQ ID NO: 12. An antibody or antigen-binding fragment thereof is provided comprising a heavy chain variable region and a light chain variable region, wherein the heavy chain variable region has an amino acid sequence comprising SEQ ID NO: 3, and wherein the light chain variable region comprises an amino acid sequence selected from SEQ ID NOS: 12, 30, 40, and 43. An antibody or antigen-binding fragment thereof is provided wherein an amino acid sequence of the heavy chain variable region can exclude SEQ. ID NO: 1, or an amino acid sequence of light chain variable region can exclude SEQ ID NO: 2, or both can be excluded.

An antibody or antigen-binding fragment thereof is provided comprising a heavy chain variable region and a light chain variable region, the heavy chain variable region having an amino acid sequence comprising an amino acid sequence at least about 80% identical to the sequence of SEQ ID NO: 3, and the light chain variable region having an amino acid sequence comprising an amino acid sequence at least about 80% identical to the sequence of SEQ ID NOS: 12 or 24, wherein the antibody or antigen-binding fragment thereof binds to deacetylated poly-N-acetyl glucosamine (dPNAG) and fewer than about 50% of the beta-1,6-linked glucosamine residues are N-acetylated, or binds to a saccharide comprising beta-1,6-linked glucosamine residues, the saccharide comprising at least five sugar residues, or binds to both.

An antibody or antigen-binding fragment thereof is provided comprising a heavy chain variable region comprising a CDRH1 sequence comprising SEQ ID NO: 4, a CDRH2 sequence comprising SEQ ID NO: 6, or a CDRH3 sequence comprising SEQ ID NO: 7, or any combination thereof; and/or a light chain variable region comprising a CDRL1 sequence comprising SEQ ID NO: 13, a CDRL2 sequence comprising the amino acid sequence Gly-Ala-Ser (GAS), or a CDRL3 sequence comprising SEQ ID NO: 16, or any combination thereof, wherein the antibody or antigen-binding fragment thereof binds to deacetylated poly-N-acetyl glucosamine (dPNAG) and fewer than about 50% of the beta-1,6-linked glucosamine residues are N-acetylated, or binds to a saccharide comprising beta-1,6-linked glucosamine residues, the saccharide comprising at least five sugar residues, or binds to both. An antibody or antigen-binding fragment thereof is provided comprising a heavy chain variable region comprising a CDRH1 sequence comprising SEQ ID NO: 4, a CDRH2 sequence comprising SEQ ID NO: 6, or a CDRH3 sequence comprising SEQ ID NO: 7, or any combination thereof; and/or a light chain variable region comprising a CDRL1 sequence comprising SEQ ID NO: 25, a CDRL2 sequence comprising the amino acid sequence GAS, or a CDRL3 sequence comprising SEQ ID NO: 27, or any combination thereof, wherein the antibody or antigen-binding fragment thereof binds to deacetylated beta-1,6-poly-N-acetyl glucosamine (dPNAG) and fewer than about 50% of the beta-1,6-linked glucosamine residues are N-acetylated, or binds to a saccharide comprising beta-1,6-linked glucosamine residues, the saccharide comprising at least five sugar residues, or binds to both. Antibodies or antigen-binding fragments thereof of the present disclosure can comprise one, two, three, or all four framework regions of any desired heavy chain region, light chain region, or both.

An antibody or antigen-binding fragment thereof can comprise a heavy chain variable region comprising an amino acid sequence of SEQ ID NO: 3, wherein the antibody or antigen-binding fragment thereof binds to deacetylated poly-N-acetyl glucosamine (dPNAG) and fewer than about 50% of the beta-1,6-linked glucosamine residues are N-acetylated, or binds to a saccharide comprising beta-1,6-linked glucosamine residues, the saccharide comprising at least five sugar residues, or binds to both. An antibody or antigen-binding fragment thereof can comprise a light chain variable region comprising an amino acid sequence of SEQ ID NO: 12, wherein the antibody or antigen-binding fragment thereof binds to deacetylated poly-N-acetyl glucosamine (dPNAG) and fewer than about 50% of the beta-1,6-linked glucosamine residues are N-acetylated, or binds to a saccharide comprising beta-1,6-linked glucosamine residues, the saccharide comprising at least five sugar residues, or binds to both. An antibody or antigen-binding fragment thereof can comprise a light chain variable region comprising an amino acid sequence of SEQ ID NO: 24, wherein the antibody or antigen-binding fragment thereof binds to deacetylated poly-N-acetyl glucosamine (dPNAG) and fewer than about 50% of the beta-1,6-linked glucosamine residues are N-acetylated, or binds to a saccharide comprising beta-1,6-linked glucosamine residues, the saccharide comprising at least five sugar residues, or binds to both.

An antibody or antigen-binding fragment thereof is provided wherein the antibody or antigen-binding fragment thereof binds a biofilm. The biofilm can comprise PNAG, or dPNAG, or both. A composition is provided that can comprise one or more antibodies or antigen-binding fragments of the present disclosure. A composition can comprise two or more different antibodies or antigen-binding fragments thereof. Such a composition can be a therapeutic or a diagnostic composition. Such compositions can be used to prevent, or treat, or both a disease, for example, one associated with a bacterial infection. A pharmaceutical composition can comprise an antibody or antigen-binding fragment, and a pharmaceutically acceptable carrier. The pharmaceutical composition can further comprise, for example, an excipient, or an adjuvant, or both. The pharmaceutical composition can be, for example, a vaccine composition. A vaccine composition can comprise, for example, an antibody or antigen-binding fragment thereof of and beta-(1,6)-poly-N-acetylglucosamine. The beta-(1,6)-poly-N-acetylglucosamine can be fully or partially deacetylated as described herein (dPNAG).

A kit comprising an antibody or antigen-binding fragment of the disclosure is provided. For example, the antibody or antigen-binding fragment in the kit can be a primary antibody or primary antigen-binding fragment thereof, and the kit can further comprise a secondary antibody or secondary antigen-binding fragment thereof that binds the primary antibody or primary antigen-binding fragment thereof.

A substrate treated with an antibody or antigen-binding fragment thereof of the disclosure is provided. The substrate can be a biological substrate, an artificial substrate, or both. The substrate can be a biological substrate, for example, a tissue or an organ. An artificial substrate can comprise, for example, a medical device or instrument. The medical device or instrument can be implantable or otherwise applied to a patient's body.

An isolated nucleic acid (polynucleotide) encoding an antibody or antigen-binding fragment thereof of the disclosure is provided. The nucleotide can be codon optimized for expression in a desired expression cell type. A plasmid comprising the nucleic acid is provided. A host cell comprising the nucleic acid is provided.

A method of administering an effective amount of the antibody or antigen-binding fragment thereof of the present disclosure to a patient in need thereof is provided. The patient can have or be at risk for a microbial infection. The microbial infection can comprise a bacterial infection, a protozoal infection, or a fungal infection, or any combination thereof. The microbial infection can be characterized by one or more bacteria that produce poly-N-acetyl glucosamine (PNAG), or deacetylated poly-N-acetyl glucosamine (dPNAG), or both. The microbial infection can be characterized by the formation of a biofilm in a patient. An antibody or antigen-binding fragment thereof can be administered alone or in combination with one or more additional therapeutic compounds, for example, a polysaccharide, a second antibody or antigen-binding fragment thereof, or an antibiotic, or any combination thereof.

A method of inhibiting formation of dPNAG-containing microbial biofilm on a substrate comprising contacting the substrate with an effective amount the antibody or antigen-binding fragment thereof disclosed herein is provided. A method of disrupting a biofilm, comprising contacting the biofilm with an effective amount of the antibody or antigen-binding fragment thereof disclosed herein is provided. A method of detecting a biofilm in a patient, comprising administering to the patient the antibody or antigen-binding fragment thereof disclosed herein, and detecting binding of the antibody or antigen-binding fragment to the biofilm is provided. A method of detecting a biofilm in a sample obtained from a patient comprising contacting the sample with the antibody or antigen-binding fragment thereof described herein is provided. A method for detecting a biofilm on a substrate is provided comprising contacting the substrate with the antibody or antigen-binding fragment thereof described herein.

### BRIEF DESCRIPTION OF THE DRAWINGS

For a further understanding of the nature, objects, and advantages of the present disclosure, reference can be made to the following detailed description, read in conjunction with the following drawings.

FIG. 1A shows: the structure of PNAG that can be found in a biofilm; FIG. 1B shows the structure of dPNAG that can be found in a biofilm; and FIG. 1C shows a representation of a biofilm including the PNAG and dPNAG from FIGS. 1A-1B respectively.

FIG. 2 depicts VH and VL chain alignments of G10, TG10, and single point mutants to the inferred germlines IGHV3-07*03 and IGKV3-20*01. The figure shows alignment of: IGHV3-07*03 (SEQ ID NO:22); G10 (SEQ ID NO:1); TG10 (SEQ ID NO:3); TG10 Y37A (SEQ ID NO:31; TG10 W38A (SEQ ID NO:47); TG10 E57A (SEQ ID NO: 37); TG10 Y66A (SEQ ID NO:38); TG10 D107A (SEQ ID NO:41); TG10 P108A (SEQ ID NO: 36); TG10 D109A (SEQ ID NO:42); TG10 D109K (SEQ ID NO:48); TG10 V110R (SEQ ID NO:39); TG10 D116A (SEQ ID NO:34).

FIG. 3A depicts TG10 binding profile at 5 μg/ml. Array profiles of TG10 and F598 are shown. Signals in relative fluorescence units (RFUs) for TG10 on an 816-component array are shown. PNAG conjugates are sorted by charge, or from highest degree of deacetylation to highest degree of acetylation. Glycan structures were created using GlycoGlyph.

FIG. 3B depicts F598 binding profile at 5 μg/ml. Array profiles of TG10 and F598. Signals in relative fluorescence units (RFUs) for TG10 on an 816-component array. PNAG conjugates are sorted by charge, or from highest degree of deacetylation to highest degree of acetylation. Glycan structures were created using GlycoGlyph.

FIG. 3C depicts binding profiles of TG10 and F598 to the 32 PNAG-BSA conjugates on the array. Array profiles of TG10 and F598. Signals in relative fluorescence units (RFUs) for TG10 on an 816-component array are shown. PNAG conjugates are sorted by charge, or from highest degree of deacetylation to highest degree of acetylation. Glycan structures were created using GlycoGlyph.

FIG. 4A depicts binding curves and apparent K_{D}'s for the top two conjugates on the array. Signals in relative fluorescence units (RFUs) for TG10 on an 816-component array. TG10 binding with the highly deacetylated PNAG conjugates PNAG 0 and PNAG 1 is depicted.

FIG. 4B depicts binding curves and apparent K_{D}'s for the top two conjugates on the array. Signals in relative fluorescence units (RFUs) for TG10 on an 816-component array are shown. F598 binding with the highly N-acetylated PNAG conjugates PNAG 30 and PNAG 31 is depicted.

FIG. 5A depicts antibody binding inhibited by monosaccharides and eliminated by a single-point mutants with TG10 binding to the fully deacetylated PNAG 0 is inhibited by 10 mM GlcNH2>GalNH2>>>GlcNAc or GalNAc.

FIG. 5B depicts antibody binding inhibited by monosaccharides and eliminated by a single point mutants with F598 binding to the fully N-acetylated PNAG31 is inhibited by 10 mM GlcNAc>>GlcNH2, GalNH2 or GlcNAc.

FIG. 5C depicts antibody binding inhibited by monosaccharides and eliminated by a single point mutants with GlcNH2 IC50 values for TG10 and the dPNAG array components PNAG 0, PNAG 1, and PNAG 16.

FIG. 5D depicts single point mutations eliminated TG10 binding with the fully deacetylated array component PNAG 0. Signals in relative fluorescence units (RFUs) for TG10 on an 816-component array are shown.

FIG. 6A depicts S. epidermidis biofilms stained with monoclonal antibodies TG10 and F598. TG10-647, F598-488, and DNA with Hoechst 33342 are differentially stained. Three channels are split with the overlay in the bottom right. Representative microcolony within the biofilm stains strongly with TG10. F598 staining is strongest on the perimeter of the microcolonies.

FIG. 6B depicts S. epidermidis biofilms stained with monoclonal antibodies TG10 and F598. TG10-647, F598-488, and DNA stained with Hoechst 33342. 3 channels are split with the overlay in the bottom right. Representative clusters of TG10 positive cells surrounds by F598 staining.

FIG. 6C depicts S. epidermidis biofilms stained with monoclonal antibodies TG10 and F598. TG10-647, F598-488, and DNA with Hoechst 33342 are differentially stained. A 3D rendering of Z-stack images is shown.

FIG. 7A depicts antibody binding to S. aureus and activity in OPK assay. Fluorescence microscopy (top) and brightfield images (bottom) of FITC labeled TG10 binding to ATCC 29213 and Xen 36 cells are shown (scale bar: 10 μm).

FIG. 7B depicts antibody binding to S. aureus and activity in OPK assay. Fluorescence microscopy (top) and brightfield images (bottom) of FITC labeled F598 binding to ATCC 29213 and Xen 36 cells are shown (scale bar: 10 μm).

FIG. 7C depicts antibody binding to S. aureus and activity in OPK assay. Confocal images of FITC-labeled F598 and 647-labeled TG10 binding to ATCC 29213 and Xen 36 cells are shown (scale bar: 2 μm).

FIG. 7D depicts antibody binding to S. aureus and activity in OPK assay. Opsonophagocytic killing (OPK) assay of TG10, F598, and mAb cocktail are shown. Killing efficiency was fitted to log total final concentration of mAbs via a 4-parameter logistic method. EC50 was calculated based on the concentration to give 50% killing.

FIG. 8A shows a confocal image of TG10 and F598. TG10-647 in, F598-488, and DNA with Hoechst 33342 are differentially stained.

FIG. 8B shows an enlarged inset of microcolony shows individual cells of the microcolony.

FIG. 9A shows confocal images of TG10 and F598 with TG10-488, F598-64, and DNA stained with Hoechst 33342 differentially stained.

FIG. 9B shows confocal images of TG10 and F598 with TG10-488.

FIG. 9C shows confocal Images of TG10 and F598 with F598-488, and DNA stained with Hoechst 33342 differentially stained.

FIG. 9D shows confocal Images of TG10 and F598 with basement, or lower areas of the biofilm show elevated TG10 staining relative to F598.

FIG. 10 show a violin plot of S. epidermidis biofilm growth inhibition. Antibodies and controls were incubated at 200 μg/mL for 8 hours, washed, and stained with crystal violet. UV absorbance at 550 nm was used to quantify crystal violet binding to PNAG.

FIG. 11 depicts of graph of Kaplan-Meier survival analysis shows effective passive protection in mouse model.

### DETAILED DESCRIPTION

The antibodies and antigen-binding fragments thereof referred to in this disclosure recite various sequences by sequence identifiers that are set forth in TABLES A-E. The organization of antibody fragments and sequence information into these five tables is arbitrary and non-limiting, and is provided for ease of reference. These tables list antibodies and fragments thereof that are also discussed in more detail in the Examples and correspond to TABLES 1-5 that appear at the beginning of the Examples. The sequences in TABLES A-E refer to variable heavy chains (HCs) and light chains (LCs) and portions thereof. Such portions include complementarity determining regions (CDRs) and framework regions (FRs). Generally, a heavy chain variable region and a light chain variable region each includes three CDRs and four FRs with the CDRs interspersed between the FRs such that an order of FR1, CDR1, FR2, CDR2, FR3, CDR3, and FR4 make up a given heavy or light chain variable region. Polypeptides including fewer than those seven regions are also within the scope of the present disclosure. Polypeptides comprising individual CDRs and/or FRs are also within the scope of the disclosure. A polypeptide can comprise a CDR alone or one or more adjacent amino acid residues on either or both sides of a CDR. Examples of sequences of such polypeptides are denoted with an “X” suffix in TABLES A-E. An “X” suffix cannot also denote extended version of an overall heavy or light chain variable region. CDRs and FRs, as well as other variable region segments, can be mixed and modified from amongst the various heavy chain variable region sequences and amongst the various light chain variable region sequences such that a paratope is formed that binds a target epitope and can provide various desirable characteristics such as enhancing opsonic killing of bacteria, for example, those found in a biofilm. All such combinations and permutations are within the scope of this disclosure. Unless otherwise indicated, acetylation refers specifically to N-acetylation of an amine group of a glucosamine residue. Similarly, deacetylation refers to removal of an acetyl group from an amine group of an N-acetyl glucosamine residue. The target epitope can be dPNAG, or PNAG, or both. That is, the target epitope can be a dPNAG with a certain percentage of deacetylation, for example, at least about 50% deacetylation such that only about 50% or less of the dPNAG remains N-acetylated. Acetylation, or deacetylation, or both can be performed naturally or artificially. For example, using an enzyme or organic synthetic chemistry. PNAG, or dPNAG, or both can be obtained from a natural or artificial source.

Variants of the polypeptides described herein are also within the scope of the present disclosure, for example, a polypeptide having an amino acid sequence that is at least about 50%, at least about 60%, at least about 75%, at least about 80% at least about 82%, at least about 85%, at least about 87%, at least about 90%, at least about 92%, at least about 93%, at least about 94%, at least about 95% at least about 96%, at least about 97%, at least about 98%, or at least about 99% identity to a sequence identified, or any intervening percent identity, or any percentage range therebetween. Residues that when mutated decrease or eliminate binding to the desired target, for example, those listed in TABLES C-E, can be preferentially retained or replaced with a more conservative mutation. Constant regions, fragments thereof, and other structures of immunoglobulins, for example, an IgG, human, humanized, or otherwise, can be paired with variable regions and portions thereof in constructing antibodies and antigen-binding fragments thereof of the disclosure. Artificial antibodies and antigen-binding fragments thereof that are distinguishable from naturally occurring antibodies and antigen-binding fragments thereof are provided by this disclosure. Uses and compositions of antibodies and antigen-binding fragments thereof, whether artificial or naturally occurring, are provided by this disclosure. Polypeptides having variable region sequences set forth herein can be paired with other polypeptides having sequences available directly from or as translations of sequences, or complements thereof, from various sequence databases in constructing antibodies and antigen-binding fragments thereof of the disclosure. Such databases include, for example GenBank, EMBL, Swiss-PROT, TREMBL, DDBJ, and the Observed Antibody Space (OAS) databases, which are incorporated by reference herein for antibody-associated sequences both generally and with respect to accession numbers referred to herein. For example, sequence information related to the 5575266 (266) light chain (LC) antibody construct can be found in the OAS database: Waltari_2018_Light_Healthy_PBMCs_Bulk_Heavy_Healthy_PBMCs_BD6_igblastn_Bulk|SRR5811776.5575266 (5575266). Similarly sequence information can be obtained or otherwise derived for construct sequences 15729 (729), 1467120 (120), 4488285 (285),

TABLE A lists sequences associated with a first group (Group I) of antibodies or antigen binding fragments thereof that can be considered “archetypes” upon which other antibodies can be based. These antibodies and fragments are discussed in more detail in the Examples section. TG10iGL HC (variable region of IGHV3-07*03) and TG10iGL LC (variable region of IGKV3-20*01). An “*” denotes an amino acid sequence in single letter code. Optimized coding sequences inclusive of sequences for ADI-45379 (G10-PNAG) HC and LC are provided in SEQ ID NOS: 52 and 53, respectively. Optimized coding sequences inclusive of sequences for TG10 HC and LC are provided in SEQ ID NOS: 54 and 55, respectively.

TABLE A

| Clone | Fragment | Sequence |
|---|---|---|
| ADI-45379 | G10 HC | SEQ ID NO: 1 |
| (G10-PNAG) | | |
| ADI-45379 | G10 LC | SEQ ID NO: 2 |
| (G10-PNAG) | | |
| TG10 | TG10 HC | SEQ ID NO. 3 |
| TG10 | TG10 HCDR1 | SEQ ID NO: 4 |
| TG10 | TG10 HCDR1X | SEQ ID NO: 5 |
| TG10 | TG10 HCDR2 | SEQ ID NO: 6 |
| TG10 | TG10 HCDR3 | SEQ ID NO: 7 |
| TG10 | TG10 HCFR1 | SEQ ID NO: 8 |
| TG10 | TG10 HCFR2 | SEQ ID NO: 9 |
| TG10 | TG10 HCFR3 | SEQ ID NO: 10 |
| TG10 | TG10 HCFR4 | SEQ ID NO: 11 |
| TG10 | TG10 LC | SEQ ID NO: 12 |
| TG10 | TG10 LCDR1 | SEQ ID NO: 13 |
| TG10 | TG10 LCDR1X | SEQ ID NO: 14 |
| TG10 | GAS* | N/A |
| TG10 | TG10 LCDR2X | SEQ ID NO: 15 |
| TG10 | TG10 LCDR3 | SEQ ID NO: 16 |
| TG10 | TG10 LCDR3X | SEQ ID NO: 17 |
| TG10 | TG10 LCFR2 | SEQ ID NO: 18 |
| TG10 | TG10 LCFR3 | SEQ ID NO: 19 |
| TG10 | TG10 LCFR4 | SEQ ID NO: 21 |
| Tg10 iGL | TG10iGL HC | SEQ ID NO: 22 |
| | (IGHV3-07*03) | |
| Tg10 iGL | TG10iGL LC | SEQ ID NO: 23 |
| | (IGKV3-20*01) | |

TABLE B lists sequences associated with a second group (Group II) of antibodies or antigen binding fragments thereof that have enhanced binding and/or specificity characteristics with respect to dPNAG. These properties can be modified as set forth in this disclosure, for example, by combining and/or modifying sequences. An denotes an amino acid sequence in single letter code. A coding sequence inclusive of that for 5575266 (266) is provided in SEQ ID NO: 56.

TABLE B

| Clone | Fragment | Sequence |
|---|---|---|
| TG × 266 | TG10HC | SEQ ID NO: 3 |
| TG × 266 | 5575266 LC | SEQ ID NO: 24 |
| TG × 266 | 5575266 LCX | SEQ ID NO: 57 |
| TG × 266 | TG × 266LCDR1 | SEQ ID NO: 25 |
| TG × 266 | TG × 266LCDR1X | SEQ ID NO: 26 |
| TG × 266 | TG × 266LCDR2 | GAS* |
| TG × 266 | TG × 266LCDR3 | SEQ ID NO: 27 |
| TG × 266 | 266 LCFR1 | SEQ ID NO: 28 |
| TG × 266 | 266 LCFR2 | SEQ ID NO: 29 |
| TG × 266 | 266 LCFR3 | SEQ ID NO: 30 |
| TG × 266 | 266 LCFR4 | SEQ ID NO: 21 |
| TG10 Y37A | TG10 HC Y37A | SEQ ID NO: 31 |
| TG10 Y37A | TG10Y37A HCDR1 | SEQ ID NO: 32 |
| TG10 Y37A | TG10Y37A | SEQ ID NO: 33 |
| | HCDR1X | |
| TG10 Y37A | TG10 LC | SEQ ID NO: 12 |
| TG10 D116A | TG10 HC D116A | SEQ ID NO: 34 |
| TG10 D116A | TGD116A HCDR3 | SEQ ID NO: 35 |
| TG10 D116A | TG10 LC | SEQ ID NO: 12 |
| TG10 P108A | TG10 HC P108A | SEQ ID NO: 36 |
| TG10 P108A | TG10 LC | SEQ ID NO: 12 |
| TG10 E57A | TG10 HC E57A | SEQ ID NO: 37 |
| TG10 E57A | TG10 LC | SEQ ID NO: 12 |

TABLE C lists sequences associated with a third group (Group III) of antibodies or antigen binding fragments thereof that have diminished binding and/or specificity characteristics with respect to dPNAG, while still retaining some binding affinity. These properties can be modified as set forth in this disclosure, for example, by combining and/or modifying sequences.

TABLE C

| Clone | Fragment | Sequence |
|---|---|---|
| TG10 Y66A | TG10 HC Y66A | SEQ ID NO: 38 |
| TG10 Y66A | TG10 LC | SEQ ID NO: 12 |
| TG10 V110R | TG10 HC V110R | SEQ ID NO: 39 |
| TG10 V110R | TG10 LC | SEQ ID NO: 12 |
| TG10 LC Y55A | TG10 HC | SEQ ID NO: 3 |
| TG10 LC Y55A | TG10 LC Y55A | SEQ ID NO: 40 |

TABLE D lists sequences associated with a fourth group (Group IV) of antibodies or antigen binding fragments thereof that have significantly diminished binding and/or specificity characteristics with respect to dPNAG, while still retaining low binding affinity. These properties can be modified as set forth in this disclosure, for example, by combining and/or modifying sequences.

TABLE D

| Clone | Fragment | Sequence |
|---|---|---|
| TG10 D107A | TG10 HC D107A | SEQ ID NO: 41 |
| TG10 D107A | TG10 LC | SEQ ID NO: 12 |
| TG10 D109A | TG10 HC D109A | SEQ ID NO: 42 |
| TG10 D109A | TG10 LC | SEQ ID NO: 12 |
| TG10 LC Y38A | TG10 HC | SEQ ID NO: 3 |
| TG10 LC Y38A | TG10 LC Y38A | SEQ ID NO: 43 |

TABLE E lists sequences associated with a fifth group (Group V) of antibodies or antigen binding fragments thereof that have essentially no binding and/or specificity characteristics with respect to dPNAG. These properties can be modified as set forth in this disclosure, for example, by combining and/or modifying sequences.

TABLE E

| Clone | Fragment | Sequence |
|---|---|---|
| 729 × TG | 15729 HC | SEQ ID NO: 44 |
| 729 × TG | TG10 LC | SEQ ID NO: 12 |
| 729 × 266 | 15729 HC | SEQ ID NO: 44 |
| 729 × 266 | 5575266 LC | SEQ ID NO: 24 |
| 120 × TG | 1467120 HC | SEQ ID NO: 45 |
| 120 × TG | TG10 LC | SEQ ID NO: 12 |
| 120 × 266 | 1467120 HC | SEQ ID NO: 45 |
| 120 × 266 | 5575266 LC | SEQ ID NO: 24 |
| 285 × TG | 4488285 HC | SEQ ID NO: 46 |
| 285 × TG | TG10 LC | SEQ ID NO: 12 |
| 285 × 266 | 4488285 HC | SEQ ID NO: 46 |
| 285 × 266 | 5575266 LC | SEQ ID NO: 24 |
| TG10 W38A | TG10 HC W38A | SEQ ID NO: 47 |
| TG10 W38A | TG10 LC | SEQ ID NO: 12 |
| TG10 D109K | TG10 HC D109K | SEQ ID NO: 48 |
| TG10 D109K | TG10 LC | SEQ ID NO: 12 |
| TG10 LC D116A | TG10 HC | SEQ ID NO: 3 |
| TG10 LC D116A | TG10 LC D116A | SEQ ID NO: 49 |

An antibody or antigen-binding fragment thereof is provided that comprises a heavy chain variable region and a light chain variable region, the heavy chain variable region having an amino acid sequence comprising SEQ ID NO: 3, and the light chain variable region having an amino acid sequence comprising SEQ ID NOS: 12 or 24. An antibody comprising a heavy chain variable region and a light chain variable region is provided; wherein the heavy chain variable region comprises an amino acid sequence selected from SEQ ID NO: 3, 31, 34, 36-39, 41, and 42; and wherein the light chain variable region comprises an amino acid sequence selected from SEQ ID NOS: 12, 24, 40, and 43. An antibody or antigen-binding fragment thereof comprising a heavy chain variable region and a light chain variable region is provided, wherein the heavy chain variable region comprises an amino acid sequence selected from SEQ ID NOS: 3, 31, 34, 36-39, 41, and 42 and wherein the light chain variable region having an amino acid sequence comprising SEQ ID NO: 12. An antibody or antigen-binding fragment thereof is provided comprising a heavy chain variable region and a light chain variable region, wherein the heavy chain variable region has an amino acid sequence comprising SEQ ID NO: 3, and wherein the light chain variable region comprises an amino acid sequence selected from SEQ ID NOS: 12, 30, 40, and 43. An antibody or antigen-binding fragment thereof is provided wherein an amino acid sequence of the heavy chain variable region can exclude SEQ. ID NO: 1, or an amino acid sequence of light chain variable region can exclude SEQ ID NO: 2, or both can be excluded.

An antibody or antigen-binding fragment thereof is provided comprising a heavy chain variable region and a light chain variable region, the heavy chain variable region having an amino acid sequence comprising an amino acid sequence at least about 80%, at least about 85%, at least about 90%, 91%, 92%, 93%, 94%, 95%, 96%, 97%, 98%, or 99% identical to the sequence of SEQ ID NO: 3, and the light chain variable region having an amino acid sequence comprising an amino acid sequence at least about 80%, at least about 85%, at least about 90%, 91%, 92%, 94%, 94%, 95%, 96%, 97%, 98%, or 99% identical to the sequence of SEQ ID NOS: 12 or 24, wherein the antibody or antigen-binding fragment thereof binds to deacetylated poly-N-acetyl glucosamine (dPNAG) and fewer than about 90%, fewer than about 75%, fewer than about 50%, fewer than about 35%, fewer than about 25%, fewer than about 10%, or fewer than about 1% of the beta-1,6-linked glucosamine residues are N-acetylated. Percent deacetylation/acetylation can be determined by any applicable technique or using any applicable instrument, for example, nuclear molecular resonance (NMR).

An antibody or antigen-binding fragment thereof is provided comprising a heavy chain variable region comprising a CDRH1 sequence comprising SEQ ID NO: 4, a CDRH2 sequence comprising SEQ ID NO: 6, or a CDRH3 sequence comprising SEQ ID NO: 7, or any combination thereof; a light chain variable region comprising a CDRL1 sequence comprising SEQ ID NO: 13, a CDRL2 sequence comprising the amino acid sequence GAS, or a CDRL3 sequence comprising SEQ ID NO: 16, or any combination thereof, wherein the antibody or antigen-binding fragment thereof binds to deacetylated poly-N-acetyl glucosamine (dPNAG) and fewer than about 90%, fewer than about 75%, fewer than about 50%, fewer than about 35%, fewer than about 25%, fewer than about 10%, or fewer than about 1% of the beta-1,6-linked glucosamine residues are N-acetylated. An antibody or antigen-binding fragment thereof is provided comprising a heavy chain variable region comprising two or more of a CDRH1 sequence comprising SEQ ID NO: 4, a CDRH2 sequence comprising SEQ ID NO: 6, and a CDRH3 sequence comprising SEQ ID NO: 7, wherein the antibody or antigen-binding fragment thereof binds to deacetylated poly-N-acetyl glucosamine (dPNAG) and fewer than about 90%, fewer than about 75%, fewer than about 50%, fewer than about 35%, fewer than about 25%, fewer than about 10%, or fewer than about 1% of the beta-1,6-linked glucosamine residues are N-acetylated. An antibody or antigen-binding fragment thereof is provided comprising a light chain variable region comprising two or more of a CDRL1 sequence comprising SEQ ID NO: 13, a CDRL2 sequence comprising the amino acid sequence GAS, and a CDRL3 sequence comprising SEQ ID NO: 16, wherein the antibody or antigen-binding fragment thereof binds to deacetylated poly-N-acetyl glucosamine (dPNAG) and fewer than about 90%, fewer than about 75%, fewer than about 50%, fewer than about 35%, fewer than about 25%, fewer than about 10%, or fewer than about 1% of the beta-1,6-linked glucosamine residues are N-acetylated. An antibody or antigen-binding fragment thereof is provided comprising a heavy chain variable region comprising two or more of a CDRH1 sequence comprising SEQ ID NO: 4, a CDRH2 sequence comprising SEQ ID NO: 6, and a CDRH3 sequence comprising SEQ ID NO: 7; and a light chain variable region comprising two or more of a CDRL1 sequence comprising SEQ ID NO: 13, a CDRL2 sequence comprising the amino acid sequence GAS, and a CDRL3 sequence comprising SEQ ID NO: 16, wherein the antibody or antigen-binding fragment thereof binds to deacetylated poly-N-acetyl glucosamine (dPNAG) and fewer than about 90%, fewer than about 75%, fewer than about 50%, fewer than about 35%, fewer than about 25%, fewer than about 10%, or fewer than about 1% of the beta-1,6-linked glucosamine residues are N-acetylated.

An antibody or antigen-binding fragment thereof is provided comprising a heavy chain variable region comprising a CDRH1 sequence comprising SEQ ID NO: 4, a CDRH2 sequence comprising SEQ ID NO: 6, and a CDRH3 sequence comprising SEQ ID NO: 7, wherein the antibody or antigen-binding fragment thereof binds to deacetylated poly-N-acetyl glucosamine (dPNAG) and fewer than about 90%, fewer than about 75%, fewer than about 50%, fewer than about 35%, fewer than about 25%, fewer than about 10%, or fewer than about 1% of the beta-1,6-linked glucosamine residues are N-acetylated. An antibody or antigen-binding fragment thereof is provided comprising a light chain variable region comprising a CDRL1 sequence comprising SEQ ID NO: 13, a CDRL2 sequence comprising the amino acid sequence GAS, and a CDRL3 sequence comprising SEQ ID NO: 16, wherein the antibody or antigen-binding fragment thereof binds to deacetylated poly-N-acetyl glucosamine (dPNAG) and fewer than about 90%, fewer than 75%, fewer than 50%, fewer than about 35%, fewer than about 25%, fewer than about 10%, or fewer than about 1% of the beta-1,6-linked glucosamine residues are N-acetylated. An antibody or antigen-binding fragment thereof is provided comprising a heavy chain variable region comprising a CDRH1 sequence comprising SEQ ID NO: 4, a CDRH2 sequence comprising SEQ ID NO: 6, and a CDRH3 sequence comprising SEQ ID NO: 7; and a light chain variable region comprising a CDRL1 sequence comprising SEQ ID NO: 13, a CDRL2 sequence comprising the amino acid sequence GAS, and a CDRL3 sequence comprising SEQ ID NO: 16, wherein the antibody or antigen-binding fragment thereof binds to deacetylated poly-N-acetyl glucosamine (dPNAG) and fewer than about 90%, fewer than about 75%, fewer than about 50%, fewer than about 35%, fewer than about 25%, fewer than about 10%, or fewer than about 1% of the beta-1,6-linked glucosamine residues are N-acetylated.

An antibody or antigen-binding fragment thereof is provided comprising a heavy chain variable region comprising a CDRH1 sequence comprising SEQ ID NO: 4, a CDRH2 sequence comprising SEQ ID NO: 6, or a CDRH3 sequence comprising SEQ ID NO: 7, or any combination thereof; a light chain variable region comprising a CDRL1 sequence comprising SEQ ID NO: 25, a CDRL2 sequence comprising the amino acid sequence GAS, or a CDRL3 sequence comprising SEQ ID NO: 27, or any combination thereof, wherein the antibody or antigen-binding fragment thereof binds to deacetylated beta-1,6-poly-N-acetyl glucosamine (dPNAG) and fewer than about 90%, fewer than about 75%, fewer than about 50%, fewer than about 35%, fewer than about 25%, fewer than about 10%, or fewer than about 1% of the beta-1,6-linked glucosamine residues are N-acetylated.

Antibodies or antigen-binding fragments thereof of the present disclosure can comprise one, two, three, or all four framework regions of any desired heavy chain region, light chain region, or both. An antibody or antigen-binding fragment thereof is provided wherein the heavy chain variable region comprises a variable region heavy chain framework 1 (HC-FR1) sequence of SEQ ID NO: 8, a HC-FR2 sequence of SEQ ID NO: 9, a HC-FR3 sequence of SEQ ID NO: 10, or a HC-FR4 sequence of SEQ ID NO: 11, or any combination thereof. An antibody or antigen-binding fragment thereof is provided wherein the light chain variable region comprises a variable region light chain framework 1 (LC-FR1) sequence of SEQ ID NO: 18, an LC-FR2 sequence of SEQ ID NO: 19, an LC-FR3 sequence of SEQ ID NO: 20; or an LC-FR4 sequence of SEQ ID NO: 21, or any combination thereof. A antibody or antigen-binding fragment thereof is provided wherein the heavy chain variable region comprises a variable region heavy chain framework 1 (HC-FR1) sequence of SEQ ID NO: 8, a HC-FR2 sequence of SEQ ID NO: 9, a HC-FR3 sequence of SEQ ID NO: 10, or a HC-FR4 sequence of SEQ ID NO: 11, or any combination thereof; and wherein the light chain variable region comprises a variable region light chain framework 1 (LC-FR1) sequence of SEQ ID NO: 18, an LC-FR2 sequence of SEQ ID NO: 19, an LC-FR3 sequence of SEQ ID NO: 20; or an LC-FR4 sequence of SEQ ID NO: 21, or any combination thereof. A antibody or antigen-binding fragment thereof is provided, wherein the heavy chain variable region comprises two or more a variable region heavy chain framework 1 (HC-FR1) sequence of SEQ ID NO: 8, a HC-FR2 sequence of SEQ ID NO: 9, a HC-FR3 sequence of SEQ ID NO: 10, and a HC-FR4 sequence of SEQ ID NO: 11; and wherein the light chain variable region comprises two or more of a variable region light chain framework 1 (LC-FR1) sequence of SEQ ID NO: 18, an LC-FR2 sequence of SEQ ID NO: 19, an LC-FR3 sequence of SEQ ID NO: 20; and an LC-FR4 sequence of SEQ ID NO: 21.

An antibody or antigen-binding fragment thereof light chain variable region can comprise a variable region light chain framework 1 (LC-FR1) sequence of SEQ ID NO: 28, an LC-FR2 sequence of SEQ ID NO: 29, an LC-FR3 sequence of SEQ ID NO: 30; or an LC-FR4 sequence of SEQ ID NO: 21, or any combination thereof. An antibody or antigen-binding fragment thereof heavy chain variable region can comprise a variable region heavy chain framework 1 (HC-FR1) sequence of SEQ ID NO: 8, a HC-FR2 sequence of SEQ ID NO: 9, a HC-FR3 sequence of SEQ ID NO: 10, or a HC-FR4 sequence of SEQ ID NO: 11, or any combination thereof; and a light chain variable region can comprise a variable region light chain framework 1 (LC-FR1) sequence of SEQ ID NO: 28, an LC-FR2 sequence of SEQ ID NO: 29, an LC-FR3 sequence of SEQ ID NO: 30; or an LC-FR4 sequence of SEQ ID NO: 21 or any combination thereof. An antibody or antigen-binding fragment heavy chain variable region can comprise two or more a variable region heavy chain framework 1 (HC-FR1) sequence of SEQ ID NO: 8, a HC-FR2 sequence of SEQ ID NO: 9, a HC-FR3 sequence of SEQ ID NO: 10, and a HC-FR4 sequence of SEQ ID NO: 11; and the light chain variable region can comprise two or more of a variable region light chain framework 1 (LC-FR1) sequence of SEQ ID NO: 28, an LC-FR2 sequence of SEQ ID NO: 29, an LC-FR3 sequence of SEQ ID NO: 30; and an LC-FR4 sequence of SEQ ID NO: 31.

An antibody or antigen-binding fragment thereof can comprise a heavy chain variable region comprising an amino acid sequence of SEQ ID NO: 3, wherein the antibody or antigen-binding fragment thereof binds to deacetylated poly-N-acetyl glucosamine (dPNAG) and fewer than about 90%, fewer than about 75%, fewer than about 50%, fewer than about 35%, fewer than about 25%, fewer than about 10%, or fewer than about 1% of the beta-1,6-linked glucosamine residues are N-acetylated. An antibody or antigen-binding fragment thereof can comprise a light chain variable region comprising an amino acid sequence of SEQ ID NO: 12, wherein the antibody or antigen-binding fragment thereof binds to deacetylated poly-N-acetyl glucosamine (dPNAG) and fewer than about 90%, fewer than about 75%, fewer than about 50%, fewer than about 35%, fewer than about 25%, fewer than about 10%, or fewer than about 1% of the beta-1,6-linked glucosamine residues are N-acetylated. An antibody or antigen-binding fragment thereof can comprise a light chain variable region comprising an amino acid sequence of SEQ ID NO: 24, wherein the antibody or antigen-binding fragment thereof binds to deacetylated poly-N-acetyl glucosamine (dPNAG) and fewer than about 90%, fewer than about 75%, fewer than about 50%, fewer than about 35%, fewer than about 25%, fewer than about 10%, or fewer than about 1% of the beta-1,6-linked glucosamine residues are N-acetylated.

An antibody or antigen-binding fragment thereof can bind to deacetylated beta-1,6-poly-N-acetyl glucosamine that is at least about 5%, at least about 10%, at least about 15%, at least about 20%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% or any percentage therebetween, or any percentage range therebetween deacetylated. An antibody or antigen-binding fragment thereof can bind to deacetylated beta-1,6-poly-N-acetyl glucosamine that is fewer than about 99%, fewer than about 95%, fewer than about 90%, fewer than about 85%, fewer than about 80%, fewer than about 75%, fewer than about 70%, fewer than about 65%, fewer than about 60%, fewer than about 55%, fewer than about 50%, fewer than about 45%, fewer than about 40%, fewer than about 35%, fewer than about 30%, fewer than about 25%, fewer than about 20%, fewer than about 15%, fewer than about 10%, fewer than about 5%, or fewer than about 1%, or any percentage therebetween, or percentage range therebetween N-acetylated. An antibody or antigen-binding fragment thereof can bind to deacetylated beta-1,6-poly-N-acetyl glucosamine that is essentially free of acetylation. An antibody or antigen-binding fragment thereof can bind to deacetylated beta-1,6-poly-N-acetyl glucosamine that is fully deacetylated. An antibody or antigen-binding fragment thereof can bind an epitope that is free of a N-acetyl moiety of N-acetyl glucosamine. For example, an antibody or antigen-binding fragment thereof can bind a pentasaccharide of beta-1,6-polyglucosamine free of acetylation, or that has a single N-acetyl moiety, or that has two N-acetyl moieties. An antibody or antigen-binding fragment thereof can bind an epitope that comprises in whole or part an N-acetyl moiety of N-acetyl glucosamine. For example, an antibody or antigen-binding fragment thereof can bind a pentasaccharide of beta-1,6-polyglucosamine that is fully N-acetylated as 1,6-poly-N-acetyl glucosamine, or that has at least three N-acetyl moieties, or that has at least four N-acetyl moieties. An antibody can have cross-reactivity between beta-1,6-polyglucosamine and beta-1,6-poly-N-acetyl glucosamine, for example, a bispecific antibody. Pentasaccharides can be free or bound, for example, to a bead or an array in assaying antibody binding.

Antibodies and antibody fragments there of can bind to various saccharides. These saccharides can vary with respect to length and composition. Saccharides can comprise one or more types of sugar residues, for example, beta-1,6,-linked glucosamine, and beta-1,6-linked N-acetyl glucosamine. The antibody or antigen-binding fragment thereof of any one of the preceding claims, wherein the saccharide comprises an oligosaccharide, or a polysaccharide, or both. Oligosaccharides can comprise, for example, from about 2 to about 20 residues, from about 3 to about 15 residues, or from about 5 to about 10 residues, or any intervening number of residues, or any range of residues therebetween. Polysaccharides can comprise more than 20 about residues, or more than about 50 residues, or more than about 100 residues, or more than about 500 residues, or more than about 1,000 residues, or more than about 5,000 residues. Oligosaccharides, polysaccharides, or both be linear, or branched, or both. Oligosaccharides, polysaccharides, or both can be cross-linked. Cross-linkers can be sugar residues, or other chemical moieties, or both. The saccharide can comprise a population of polysaccharides differing in number of beta-1,6-linked glucosamine residues free of N-acetylation and number of beta-1,6-linked N-acetyl glucosamine residues. The saccharide can comprise a population of polysaccharides differing in percent content of beta-1,6-linked glucosamine residues free of N-acetylation and percent content of beta-1,6-linked N-acetyl glucosamine residues.

The antibody or antigen-binding fragment thereof can, for example, bind to a pentasaccharide comprising four beta-1,6-linked glucosamine residues free of N-acetylation, and one beta-1,6-linked N-acetyl glucosamine residue. The antibody or antigen-binding fragment thereof can, for example, bind to a pentasaccharide comprising five beta-1,6-linked glucosamine residues free of N-acetylation. The beta-1,6-linked N-acetyl glucosamine residue can, for example, be an internal residue or a terminal residue. The antibody or antigen-binding fragment thereof can, for example, bind to a pentasaccharide comprising three beta-1,6-linked glucosamine residues free of N-acetylation, and two beta-1,6-linked N-acetyl glucosamine residues. The pentasaccharide can comprise two or three adjacent beta-1,6-linked glucosamine residues free of N-acetylation. The antibody or antigen-binding fragment thereof can, for example, bind to a pentasaccharide comprising two beta-1,6-linked glucosamine residues free of N-acetylation, and three beta-1,6-linked N-acetyl glucosamine residues. The pentasaccharide can comprise two adjacent or non-adjacent beta-1,6-linked glucosamine residues free of N-acetylation. The saccharide can be other than a pentasaccharide, for example, oligosaccharides and polysaccharides of different number of residues comprising such sequences of five residues.

An antibody or antigen-binding fragment thereof can bind to at least about 50% deacetylated dPNAG with a dissociation constant (K_{D}) of less than about 5×10⁻⁸ M, less than about 1×10⁻⁸ M, less than about 5×10⁻⁹ M, less than about 1×10⁻⁹ M, less than about 5×10⁻¹⁰ M, less than about 1×10⁻¹⁰ M, less than about 5×10⁻¹¹ M, less than about 1×10⁻¹¹ M, less than about 5×10⁻¹² M, or less than about 1×10⁻¹² M, for, example, as measured by biolayer interferometry. An antibody or antigen-binding fragment of the present disclosure can compete for binding with PNAG, dPNAG, or both with another antibody. An antibody or antigen-binding fragment thereof can compete, for example, with monoclonal antibody F598 in binding to dPNAG.

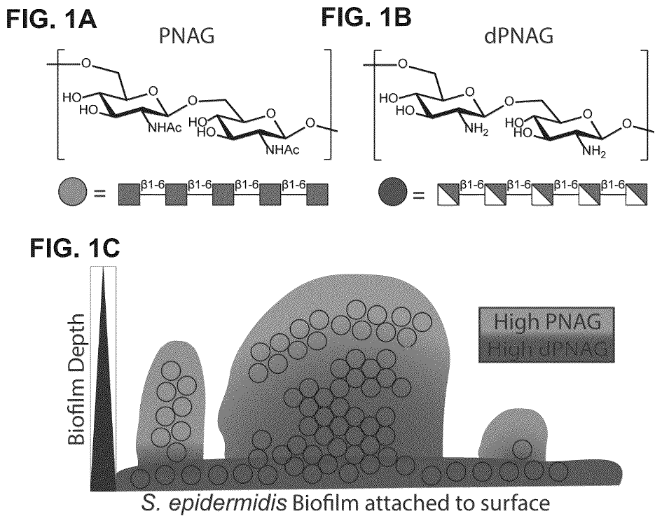

An antibody or antigen-binding fragment thereof is provided wherein the antibody or antigen-binding fragment thereof binds a biofilm. The biofilm can comprise PNAG, or dPNAG, or both. FIG. 1A shows the structure of PNAG that can be found in a biofilm. FIG. 1B shows the structure of dPNAG that can be found in a biofilm. FIG. 1C shows a representation of a biofilm including the PNAG and dPNAG from FIGS. 1A and 1B respectively. The dPNAG of the biofilm can on average be from about 1% to about 99%, from about 5% to about 95%, from about 10% to about 90%, from about 15% to about 85%, from about 20% to about 80%, from about 25% to about 75%, from about 30% to about 70%, from about 35% to about 65%, from about 40% to about 60%, from about 45% to about 55%, at least about 99%, at least about 90%, at least about 85%, at least about 75%, or at least about 50%, or any intervening percentage, or any intervening range, or deacetylated, or fully deacetylated. The biofilm can comprise one or more bacteria. The one or more bacteria can produce the biofilm. The one or more bacteria can produce PNAG, or dPNAG, or both. The one or more bacteria can produce sufficient dPNAG to promote formation of a biofilm. The antibody or antigen-binding fragment thereof can promote opsonic killing of one or more bacteria.

An antigen-binding fragment can be, for example, a Fab, Fv, scFv, Fab′, or (Fab′)2, or any combination thereof. An antibody or antibody fragment can comprise a full-length heavy chain, or a full-length light chain, or both. An antibody or antibody fragment can comprise an IgM antibody, an IgD antibody, an IgG antibody, an IgE antibody, or an IgA antibody, or any immunological fragment thereof, or any combination thereof. An antibody or antibody fragment can comprise a constant region of an IgM antibody, an IgD antibody, an IgG antibody, an IgE antibody, or an IgA antibody, or any immunological fragment thereof, or any combination thereof. An antibody or antibody fragment can comprise an IgM antibody, an IgD antibody, an IgG antibody, an IgE antibody, or an IgA antibody, or any combination thereof. An antibody or antibody fragment can comprise an IgG1 antibody, an IgG2 antibody, an IgG3 antibody, an IgG4 antibody, or any immunological fragment thereof, or any combination thereof. An antibody or antibody fragment can comprise a constant region of an IgG1 antibody, an IgG2 antibody, an IgG3 antibody, an IgG4 antibody, or any immunological fragment thereof, or any combination thereof.

An antibody or antigen-binding fragment thereof can comprise an isolated antibody or antigen-binding fragment thereof. An antibody or antigen-binding fragment thereof can comprise polyclonal antibodies or antigen-binding fragments thereof. An antibody or antigen-binding fragment thereof can comprise a monoclonal antibody or an antigen-binding fragment thereof. An antibody or antigen-binding fragment thereof can comprise a chimeric antibody or an antigen-binding fragment thereof. An antibody or antigen-binding fragment thereof can comprise a human antibody, a humanized antibody, or an antigen-binding fragment thereof, or any combination thereof. An antibody or antigen-binding fragment thereof, wherein the antibody comprises a caninized antibody, a felinized antibody, or an equinized antibody, or any combination thereof. A “species” antibody, for example a “human” antibody, refers to an antibody that possesses one or more identifying characteristics of an antibody that is produced by the specified species, but does not mean that such a species antibody is actually produced by or is otherwise naturally occurring in that or any other species (organism). A “species” antibody can differ by one or more amino acid residues or other modifications from an antibody that naturally occurs in the species. A “species” “ized” antibody can refer to an antibody that was derived from one or more species and modified to possess characteristics associated with antibodies from a different species. For example, a murine antibody can be “humanized” to possess characteristics identifiable with an antibody produced in a human. An antibody or antigen-binding fragment thereof can comprise a bispecific antibody or an antigen-binding fragment thereof. For example, a bispecific (bispecific) antibody or antigen-binding fragment thereof can comprise two different specificities to dPNAG, for example, as measured as a dissociation constant or with respect to a preference for binding to a particular percentage of deacetylation. A bispecific antibody or antigen-binding fragment thereof can comprise specificity to dPNAG and to a second antigen.

An antibody or antigen-binding fragment thereof can be conjugated to one or more detectable labels. For example, the one or more detectable labels can comprise a fluorescent label, a colorimetric label, an enzyme-linked label, or a radiolabel, or any combination thereof. An antibody or antigen-binding fragment thereof can be conjugated to a therapeutic molecule. An antibody or antigen-binding fragment thereof can be conjugated to an immunomodulator, a cytokine, a cytotoxic agent, a chemotherapeutic agent, a diagnostic agent, an antiviral agent, an antimicrobial agent, or a drug, or any combination thereof. An antibody or antigen-binding fragment thereof can be conjugated to one or more moieties to affect its pharmacokinetics or minimize a host-raised immune response against it. For example, an antibody or antigen-binding fragment can be pegylated, or have its glycosylation profile modified, or both. An antibody or antigen-binding fragment thereof can be modified to aid in its purification. For example, a histidine tag can be added to aid in purification by column chromatography.

A composition is provided that can comprise one or more antibodies or antigen-binding fragments of the present disclosure. A composition can be diagnostic, therapeutic, or both. The composition can be a liquid. The composition can be aqueous. The composition can be lyophilized. The composition can comprise one or more additional components. For example, the composition can comprise a stabilizer, a buffer, a solvent, a preservative, a chelator, a colorant, or a suspending agent, or any combination thereof. An antibody or antigen-binding fragment thereof can be present in a composition at less than about 1.0 ng/ml, from about 1.0 ng/ml to about 500 mg/mL, from about 10 ng/ml to about 100 mg/mL, from about 100 ng/ml to about 1.0 mg/mL, from about 1.0 μg/mL to about 100 μg/mL, or more than 500 mg/mL, or any intervening concentration, or any range therebetween.

A composition can comprise two or more different antibodies or antigen-binding fragments thereof. Such a composition can be a therapeutic or a diagnostic composition. A composition can comprise an antibody or antigen-binding fragment in combination with a second antibody or antigen-binding fragment thereof. The second antibody or antigen-binding fragment thereof can bind to, for example, PNAG, or dPNAG, or both. The second antibody or antigen-binding fragment thereof can bind to a polysaccharide, for example, a polysaccharide other than PNAG or dPNAG. The second antibody or antigen-binding fragment thereof can differ from the first with respect to specificities to dPNAG, for example, as measured as a dissociation constant or with respect to a preference for binding to a particular percentage of deacetylation. For example, a composition can combine a first antibody favoring binding to dPNAG that is at least about 50% deacetylated with a second antibody favoring binding to dPNAG that is less than about 50% deacetylated. The second antibody or antigen-binding fragment thereof can bind to a biofilm. The second antibody or antigen-binding fragment thereof can be produced from a deposited hybridoma having Accession No. PTA-5931 (F598), described in U.S. Patent Application Publication No. 2012/0201834, which is incorporated by reference in its entirety herein. The second antibody or antigen-binding fragment thereof can comprise one that binds a target in a biofilm other than PNAG or dPNAG. For example, the second antibody or antigen-binding fragment thereof can be an antibody or antigen-binding fragment thereof that binds a DNA binding II (DNABII) protein such as that described in U.S. Patent Application Publication No. 2021/0206841, which is incorporated by reference in its entirety herein. Action against a biofilm, measured, for example, using an opsonophagocytic killing (OPK) assay, between two or more different antibodies or fragments thereof can be additive or synergistic.

A pharmaceutical composition can comprise an antibody or antigen-binding fragment, and a pharmaceutically acceptable carrier. The pharmaceutical composition can further comprise, for example, an excipient, or an adjuvant, or both. The pharmaceutical composition can be, for example, a vaccine composition. A vaccine composition can comprise, for example, an antibody or antigen-binding fragment thereof of and beta-(1,6)-poly-N-acetylglucosamine. The beta-(1,6)-poly-N-acetylglucosamine can be fully or partially deacetylated as described herein (dPNAG). Partially deacetylated beta-(1,6)-poly-N-acetylglucosamine can be considered as a copolymer of beta-1,6-linked glucosamine residues and beta-1,6-linked-N-acetyl glucosamine residues. Such a copolymer can differ with respect to the numbers of those two residues and their relative arrangement. For example, a linear saccharide can contain stretches of primarily or exclusively one of type of residues, or stretches of mixtures of residue types, or both. Branched saccharides can also have such arrangements. One or more additional types of sugar residues can also be present in the saccharide.

A kit comprising an antibody or antigen-binding fragment of the disclosure is provided. For example, the antibody or antigen-binding fragment in the kit can be a primary antibody or primary antigen-binding fragment thereof, and the kit can further comprise a secondary antibody or secondary antigen-binding fragment thereof that binds the primary antibody or primary antigen-binding fragment thereof.

A substrate treated with an antibody or antigen-binding fragment thereof of the disclosure is provided. The substrate can be a biological substrate, an artificial substrate, or both. The substrate can be a biological substrate, for example, a tissue or an organ. A biological substrate can comprise a solid tissue, or a soft tissue, or both. The solid tissue can comprise a tooth, bone, hair, or nail, or any combination thereof. The soft tissue can comprise an internal or external tissue. The soft tissue can comprise an epidermis. The soft tissue can comprise a wound. With respect to a patient, human or otherwise, the biological substrate can comprise an endogenous tissue, an exogenous tissue, or both. The biological substrate can comprise a transplant tissue or organ. For example, the biological substrate can comprise a autograft, an allograft, or a xenograft, or any combination thereof. The biological substrate can comprise live cells, dead cells, or both. The biological substrate can comprise a consumable, for example, a food, a beverage, or both. The food or beverage can comprise a meat, a vegetable, a fruit, a juice, a dairy product, a cereal, or a product derived from any thereof, or any combination thereof.

An artificial substrate can comprise, for example, a medical device or instrument. The medical device or instrument can be implantable or otherwise applied to a patient's body. The medical device or instrument can be configured for temporary or permanent insertion into or application on a patient's body. The medical device or instrument can be applied to the surface of a patient's body or into any orifice of the patient's body, for example, an oral cavity, a nasal cavity, an ear canal, an anus, a penis, or a vagina, or any combination thereof. A medical instrument can comprise, for example, a scalpel, a forceps, a sponge, a mesh, a hose, a lighting device, an endoscope, or a laparoscopic device, or any combination thereof. The artificial substrate can comprise, for example, a surgical implant, a dental implant, a nasal implant, an optical implant, or a dermally applied device, a prosthesis, or any combination thereof. The artificial substrate can comprise, for example, a mask or other device fitted over, or into, or both of the nose, the mouth, or both. For example, such a device can comprise a continuous positive airway pressure (CPAP) device, a respirator, a ventilator, a filtering mask, or any combination thereof. A dermally applied device can comprise a bandage or other dressing. The surgical implant can comprise, for example, a stent, a catheter, a cannula, an artificial joint, a prosthesis, or a pace-maker, or any combination thereof.

An isolated nucleic acid (polynucleotide) encoding an antibody or antigen-binding fragment thereof of the disclosure is provided. The nucleotide can be codon optimized for expression in a desired expression cell type. A plasmid comprising the nucleic acid is provided. A host cell comprising the nucleic acid is provided. The host can be an isolated host cell. The host cell can a prokaryotic cell or an eukaryotic cell. The host cell can be a bacterial cell, a fungal cell, or a mammalian cell. The host cell can be, for example, a human cell. The human cell can be in vitro or in vivo. The host cell can be configured for production of antibodies engineered for a desired species, for example, humans. The host cell can be engineered for desired processing of the antibody, for example, glycosylation. The host cell can be configured for growth in a cell culture. The cell culture can be configured for growth in solution, or on a substrate, or both. The cell culture can be provided in a bioreactor. A method of producing an antibody or antigen-binding fragment thereof is provided comprising culturing the host cell and isolating the antibody or antigen-binding fragment thereof.

A method of administering an effective amount of the antibody or antigen-binding fragment thereof of the present disclosure to a patient in need thereof is provided. The patient can have or be at risk for a microbial infection. The microbial infection can comprise a bacterial infection, a protozoal infection, or a fungal infection, or any combination thereof. The microbial infection can be characterized by one or more bacteria that produce poly-N-acetyl glucosamine (PNAG), or deacetylated poly-N-acetyl glucosamine (dPNAG), or both. The microbial infection can be characterized by the formation of a biofilm in a patient. The biofilm can be characterized by poly-N-acetyl glucosamine (PNAG), or deacetylated poly-N-acetyl glucosamine (dPNAG), or both. The antibody or antigen-binding fragment thereof can be administered to provide passive immunity to the patient.

The method can be performed on a patient who has had, is undergoing, or is scheduled to undergo a surgical procedure. The surgical procedure can comprise implantation of a structure susceptible for formation of a biofilm. The surgical procedure can comprise the installation of a surgical implant in the patient. The surgical implant can comprise a stent, a catheter, a cannula, an artificial joint, a prosthesis, or a pace-maker, or any combination thereof. The patient can be a mammal. For example, the patient can be a human, a mouse, a dog, a cat, a horse, a pig, a cow, a sheep, or a goat.

A patient treated using a method disclosed herein can have any treatable microbial infection or combination of infections. For example, the microbial infection can comprise a lung infection, a joint infection, an endocardial infection, a skin infection, a soft tissue infection, septicemia, endocarditis, osteomyelitis, otitis media, periodontitis, or any combination thereof. The microbial infection can comprise, for example, one or more bacteria of the genus Staphylococcus. The one or more staphylococcal bacteria can comprise, for example, S. epidermidis, or S. aureus, or both. The S. aureus bacterium can be a methicillin-resistant S. aureus. The microbial infection can comprises one or more bacteria selected from E. coli, Yersinia pestis, Yersinia entercolitica, Xanthomonas axonopodis, Pseudomonas fluorescens, Actinobacillus actinomycetemcomitans, Actinobacillus pleuropneumoniae, Ralstonia solanacearum, Bordetella pertussis, Bordetella parapertussis, and Bordetella bronchiseptica.

The antibody or antigen-binding fragment thereof can be administered at a dose of less than about 0.1 mg/kg, from about 0.1 mg/kg to about 100 mg/kg about from 0.5 mg/kg to about 75 mg/kg, from about 1.0 mg/kg to about 50 mg/kg, from about 5.0 mg/kg to about 25 mg/kg, from about 10 mg/kg to about 20 mg/kg, from about 12 mg/kg to about 16 mg/kg, or greater than about 100 mg/kg, or any value intervening dose, or any dosage range therebetween, or any combination thereof. The amount of antibody or antigen-binding fragment thereof per dose can be less than about 0.1 mg, from about 0.1 mg to about 1,000 mg, from about 1.0 mg to about 900 mg, from about 10 mg to about 750 mg, from about 50 mg to about 500 mg, from about 100 mg to about 350 mg, from about 150 mg to about 250 mg, or greater than 1,000 mg, or any intervening amount, or any range therebetween. The administered dose can achieve a serum level of anti-dPNAG antibody of less than about 1.0 μg/ml, from about 1.0 μg/mL to about 100 μg/ml, greater than about 100 μg/ml, from about 5.0 μg/mL to about 75 μg/ml, from about 10 μg/mL to about 50 μg/ml, greater than about 100 μg/mL greater than about 100 μg/mL or any intervening serum level, or any serum range therebetween, or any combination thereof. The opsonic activity of the patient's serum can remain essentially constant, for example, for less than 5 days, from about 5 days to about 150 days, from about 10 days to about 100 days, from about 25 days to about 75 days, or more than 150 days, or any intervening duration, or any duration range therebetween after administration of the antibody. The antibody or antigen-binding fragment thereof can have a serum half-life, for example, for less than a day, from about 1 day to about 100 days, from about 10 days to about 75 days, from about 25 days to about 50 days, or more than 150 days, or any intervening duration, or any duration range therebetween after administration of the antibody.

The antibody or antigen-binding fragment thereof can be administered using any appropriate number of doses. The antibody or antigen-binding fragment can be administered as a single dose or as multiple doses. Multiple doses can be, for example, two, three, four, five, six, seven, eight, nine, or ten doses, or a higher number of doses. The antibody or antigen-binding fragment thereof can be administered once a day, every other day, every three days, every week, biweekly, every month, every six weeks, every two months, every three months, every six months, or once a year, or less frequently than a period thereof, or more frequently than a period thereof, or for a period therebetween. The dosing and scheduling of at least one dose can be based upon, for example, a determination of the antibody's or antibody binding fragment thereof's serum half-life in the patient. The dosing and scheduling of at least one dose can be based upon, for example, a determination of the in vitro opsonic activity of the patient's serum against PNAG-expressing bacteria. A method of administration can further comprise determining an effective serum titer of the administered antibody using an in vitro opsonophagocytosis assay.

The antibody or antigen-binding fragment thereof can be administered using any appropriate route, for example, by intravenous infusion. The intravenous infusion can be administered over from about 10 minutes to about 3 hours, from about 20 minutes to about 150 minutes, from about 30 minutes to about 2 hours, or from about 45 minutes to about 90 minutes, for any intervening duration. The intravenous infusion volume can be less than about 1.0 mL, from about 1.0 mL to about 500 mL, from about 10 mL to about 250 mL, from about 50 mL to about 125 mL, about 100 ml, or more than about 500 mL, or any intervening volume, or volumetric range therebetween. The antibody or antigen-binding fragment can be administered systemically. The antibody or antigen-binding fragment can be administered locally, for example, locally to the site of a wound, body cavity, or an implanted medical device. The antibody or antigen-binding fragment thereof is applied to a medical device before implantation, during implantation, or after implantation, or any combination thereof. The antibody or an antigen-binding fragment thereof is administered in a controlled release formulation.

An antibody or antigen-binding fragment thereof can be administered alone or in combination with one or more additional therapeutic compounds. A method of administering the antibody or antigen-binding fragment thereof can further comprise administering one or more polysaccharides. A polysaccharide administered can have an average molecular weight of less than about 10 kDa, from about 10 to about 1,000 kDa, more than about 1,000 kDa, from about 50 to about 750 kDa, from about 100 to about 500 kDa, from about 150 to about 250 kDa, more than about 1,000 kDa, or any intervening molecular weight thereof, or any molecular weight range thereof. Beta-1,6-poly-N-acetyl glucosamine (PNAG), partially deacetylated beta-1,6-poly-N-acetyl glucosamine (dPNAG), or fully deacetylated dPNAG, or intervening dPNAG as described therein, for example, at least about 50% deacetylated, can be administered. A method can further comprise administering one or more adjuvant.

A method can further comprise administering a second antibody or antigen binding fragment thereof. The second antibody or antigen-binding fragment thereof administered can be produced from a deposited hybridoma having Accession No. PTA-5931 (F598), or otherwise produced to have an equivalent structure, described in U.S. Patent Application Publication No. 2012/0201834, which is incorporated by reference in its entirety herein. The second antibody or antigen-binding fragment thereof can comprise one that binds a target in a biofilm other than PNAG or dPNAG. For example, the second antibody or antigen-binding fragment thereof can be an antibody or antigen-binding fragment thereof that binds a DNA binding II (DNABII) protein such as that described in U.S. Patent Application Publication No. 2021/0206841, which is incorporated by reference in its entirety herein. Inhibition of a microbial infection in a patient by administration of two or more different antibodies or fragments thereof can be additive or synergistic.

A method can further comprise administering one or more antibiotics. For example, one or more other antibody or antigen-binding fragments thereof can be administered, or one or more small molecule antibiotics, or both. For example, co-administration can be performed with a penicillin, a tetracycline, a cephalosporin, a fluoroquinolone, a lincomycin, a macrolide, a glycopeptide, an aminoglycoside, or a carbapenem, or any combination thereof. Co-administration can be performed with amoxicillin, doxycycline, cephalexin, ciprofloxacin, clindamycin, metronidazole, azithromycin, sulfamethoxazole, trimethoprim, clavulanate, or levofloxacin, or any combination thereof. Co-administration with an antibiotic can result in additive or synergistic inhibition of a microbial infection in a patient.

A method of inhibiting formation of dPNAG-containing microbial biofilm on a substrate comprising contacting the substrate with an effective amount the antibody or antigen-binding fragment thereof disclosed herein is provided. A method of disrupting a biofilm, comprising contacting the biofilm with an effective amount of the antibody or antigen-binding fragment thereof disclosed herein is provided. A method of detecting a biofilm in a patient, comprising administering to the patient the antibody or antigen-binding fragment thereof disclosed herein, and detecting binding of the antibody or antigen-binding fragment to the biofilm is provided. A method of detecting a biofilm in a sample obtained from a patient comprising contacting the sample with the antibody or antigen-binding fragment thereof described herein is provided. A method for detecting a biofilm on a substrate is provided comprising contacting the substrate with the antibody or antigen-binding fragment thereof described herein. Any of these methods can employ additional efficacious agents for example, one or more different antibodies or antigen-binding fragments thereof, or one or more antibiotics, or both.

### EXAMPLES

Tables 1-5 summarize antibodies and mutants thereof relevant to following examples and the disclosure generally. Tables 1-5 correspond to Tables A-E presented above with respect to the grouping and listing of antibody clones. The third column of each table entitled “PNAG Binding on Array” refers to the ability to bind both dPNAG and PNAG and intermediate levels of deacetylation. A complete knockdown for a particular mutant does not mean that every amino acid substitution at that position will be completely inactive. For example, whereas D109K might result in a loss of binding, but D109E might result in no or minimal binding changes because it is a relatively conservative mutation.

TABLE 1

| | | PNAG | |
|---|---|---|---|
| | | Binding on | Effect of |
| Clone | Mutation | Array | Mutation |
| ADI-45379 | Original | Yes | N/A |
| (G10-PNAG) | clone from | | |
| | screen | | |
| TG10 | Primer | Yes | N/A |
| | mutations in | | |
| | ADI-45379 | | |
| | reverted to | | |
| | germline | | |
| Tg10 iGL | TG10 | NO | N/A |
| | Inferred | | |
| | germline | | |

TABLE 2

| | | PNAG | |
|---|---|---|---|
| | | Binding on | |
| Clone | Mutation | Array | Effect of Mutation |
| TG × 266 | TG10 HC | Yes, Higher | Productive clone |
| | 5575266 LC | Selectivity | with high |
| | | for dPNAG | selectivity for |
| | | than TG10 | dPNAG. Displays |
| | | | biofilm binding |
| TG10 | TG10 HC | Yes | Modest increase |
| Y37A | Y37A | | in dPNAG |
| | | | selectivity. |
| TG10 | TG10 HC | Yes | Modest increase |
| D116A | D116A | | in dPNAG |
| | | | selectivity |
| TG10 | TG10 HC | Yes | Minimal increase |
| P108A | P108A | | in dPNAG |
| | | | selectivity. |
| TG10 | TG10 HC | Yes | Altered selectivity |
| E57A | E57A | | vs TG10 |

TABLE 3

| | | PNAG | |
|---|---|---|---|
| | | Binding on | |
| Clone | Mutation | Array | Effect of Mutation |
| TG10 | TG10 HC | Yes | Moderately |
| Y66A | Y66A | | reduced binding |
| | | | to array |
| | | | conjugates |
| TG10 | TG10 HC | Yes | Decreased affinity |
| V110R | V110R | | for PNAG array |
| | | | conjugates |
| TG10 LC | TG10 LC | Yes | Decreased affinity |
| Y55A | Y55A | | for PNAG array |
| | | | conjugates |

TABLE 4

| | | PNAG | |
|---|---|---|---|
| | | Binding on | |
| Clone | Mutation | Array | Effect of Mutation |
| TG10 | TG10 HC | Minimal | Near complete |
| D107A | D107A | binding on | knockdown of |
| | | the array | PNAG binding |
| TG10 | TG10 HC | Significant | Significant |
| D109A | D109A | reduction of | knockdown |
| | | PNAG | |
| | | binding on | |
| | | the array | |
| TG10 LC | TG10 LC | Significant | Significant |
| Y38A | Y38A | reduction of | knockdown |
| | | PNAG | |
| | | binding on | |
| | | the array | |

TABLE 5

| | | PNAG | |
|---|---|---|---|
| | | Binding on | |
| Clone | Mutation | Array | Effect of Mutation |
| 729 × TG | 15729 HC | No | No binding |
| | TG10 LC | | observed |
| 729 × 266 | 15729 HC | No | No binding |
| | 5575266 LC | | observed |
| 120 × TG | 1467120 HC | No | No binding |
| | TG10 LC | | observed |
| 120 × 266 | 1467120 HC | No | No binding |
| | 5575266 LC | | observed |
| 285 × TG | 4488285 HC | No | No binding |
| | TG10 LC | | observed |
| 285 × 266 | 4488285 HC | No | No binding |
| | 5575266 LC | | observed |
| TG10 | TG10 HC | No | Complete |
| W38A | W38A | | knockdown |
| TG10 | TG10 HC | No | Complete |
| D109K | D109K | | knockdown |
| TG10 LC | TG10 LC | No | Complete |
| D116A | D116A | | knockdown of |
| | | | PNAG binding |

### Example 1

This example describes the identification of an antibody and variants thereof having desirable properties including, for example, selective binding to dPNAG, superior to existing antibodies. A library of human-derived antibodies was screened on an 816-component array. One of the anti-glycan antibodies identified in that screen displayed highly selective binding to dPNAG. A detailed analysis of the carbohydrate recognition, the biofilm binding and inhibition, and the opsonic killing activity of an antibody TG10, modified from the human-derived antibody identified in the original screen, is provided. Remarkably, the antibody TG10 displays higher selectivity for fully deacetylated PNAG, unique biofilm binding, and improved opsonic killing when compared to a previously identified antibody (F598).

Neoglycoprotein (NGP) microarray fabrication was performed as follows. Several NGP microarrays were constructed as previously described. Two large arrays (8 duplicate arrays per slide) composed of 815 and 870 neoglycoproteins, glycoproteins, glycopeptide conjugates, and controls were constructed and used for the experiments in this report. Additionally, a PNAG-focused array (16 duplicates per slide) with 49 array components was fabricated and used to determine apparent K_{D} values. Array component parent stocks were diluted to 125-200 μg/mL into print buffer (1×PBS buffer with 2.5% (v/v) glycerol, 0.0005% (v/v) Triton-X 100, 0.0005 μg/mL soluble print dye 0.05 μg/mL Atto 532 (Sigma 06699). Formulated components were printed in duplicate onto 2D-Epoxy functionalized glass slides (Poly-An #104-00-221) or SuperEpoxy 2 microarray slides (ArrayIt) using an Arrayit NanoPrint LM60 arrayer at 60% humidity. Following the print, slides were scanned in a microarray fluorescence scanner (Innopsys, InnoScan 1100 AL) to validate that array components were successfully deposited onto the slide surface. Binding profiles for a representative set of lectins and antibodies were used as QC for each printed microarray batch. The printed NGP microarrays were stored under vacuum at −20° C.

Antibody expression and purification were performed as follows. Antibodies were obtained from transient transfection in Expi293 cells. Antibody expressions were carried out by Genscript. Plasmid inserts were synthesized and cloned into expression plasmids by Genscript (Piscataway, NJ). Plasmid inserts were preceded by the signal sequence for human IgG, amino acid (SEQ ID NO: 50) and coding (SEQ ID NO: 51). Plasmids for heavy chain (pFuse-CHIg-hG1) and kappa light chain (pFuse-CLIg-hk) were obtained from Invivogen. ElectroMAX DH10B cells (Thermo) were transformed by electroporation and grown on antibiotic resistance plates. Single colonies were picked and expanded sequentially to 100 mL cultures (TB). 100 mL overnight cultures were pelleted by centrifugation (15 min@3200 g) and the plasmid DNA was extracted and purified by maxiprep (Zymo Research). Sequences were confirmed by Sanger sequencing.

Expi293 cells were cultured in Expi293 serum free media. For a 30 mL expression, 75 million cells were transfected using 80 μL expifectamine precomplexed with 15 μg of each the heavy and the light chain plasmids in 3 mL opti-mem SFM. Newly transfected cells were incubated in 125 mL flasks at 37° C. and 8% CO₂ with 125 rpm shaking. Expi expression additives were added after 16-18 hours according to manufacturer's recommended protocol. Cultures were harvested after 6-7 days. Cells and cellular debris were removed by centrifugation, and media was cleared by sequential filtration through 3.2 μm, 1.6 μm and 0.45 μm syringe filters. The cleared media was diluted 1:1 with Protein A binding buffer (PBS, pH 7.0+0.02% NaN₃ w/v). 250 UL Protein A Resin (Genscript) was added to the buffered media. The mixture was incubated at RT with rotation for 3 hours. Media/Resin was loaded onto an Econopac column, and washed 4×5 mL with Protein A binding buffer. Recombinantly expressed mAbs were eluted from the resin using 100 mM glycine pH 3.0 and immediately neutralized with Tris pH 8.0. Eluted antibodies were concentrated in amicon spin filters and buffer-exchanged into PBS pH 7.4. Protein A purified mAbs were then loaded onto an SEC column (Superdex 200 Increase 10/300 GL, Cytiva Life Sciences) for FPLC SEC purification. Antibody purity was determined by analytical HPLC SEC chromatography.

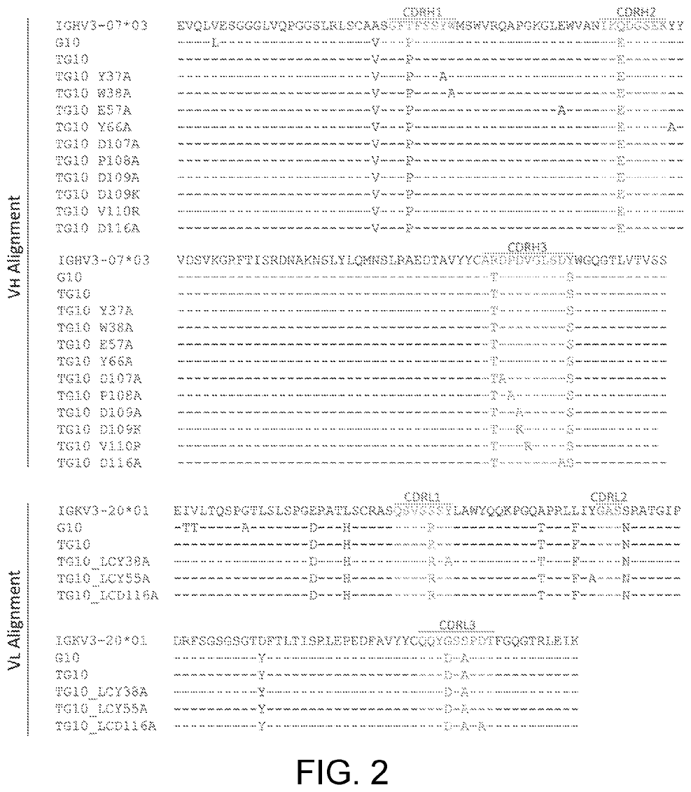

TG10 sequence and germline usage was determined to be unrelated to F598. FIG. 2 shows VH and VL chain alignments of G10, TG10, and single point mutants to the inferred germlines IGHV3-07*03 and IGKV3-20*01. The sequence of the dPNAG-binding antibody, ADI-45379, was analyzed using the IgBlast tool from NCBI. As shown in FIG. 2, the inferred germline V genes used by ADI-45379 were IGHV3-07*03 (97.3% identity) and IGKV3-20*01 (96.0% identity), and the J genes were IGHJ04*02 and IGKJ05*01, respectively. ADI-45379 heavy and light chain sequences had 6 and 12 amino acid mutations relative to the closest germline sequences. These differences included several mutations in the N-terminal regions of both the heavy and the light chains that might have originated from primers used in PCR amplification of the variable regions from the original B cell. These putative mutations were changed to mirror a germline sequence in designing TG10, bearing 5 amino acid mutations in the heavy chain and 9 amino acid mutations in the light chain due to their perceived rarity. For the heavy chain, glutamic acid occurs at position 58 at a frequency of <1% in human antibodies, and proline occurs at position 29 at a frequency of 1%. For the light chain, histidine at position 21, phenylalanine at position 53, and tyrosine at position 86 all occur at a frequency of <1%.

The resulting sequence of TG10 has a sequence that differs from known human antibodies. For example, comparing TG10 to F598, the two antibodies have little in common. The two antibodies use completely different V genes, resulting in only 52% homology between the heavy chains and 45% homology between the kappa light chain of TG10 and the lambda light chain of F598. Furthermore, the HC CDR3 lengths vary significantly: 11 amino acids for TG10 and 18 amino acids for F598. To broaden the analysis of potentially related antibodies, the Observed Antibody Space (OAS) database was searched, which contained about 430 million human heavy chains and 55 million human light chains at the time. For the TG10 heavy chain, the closest match had 95.5% nucleotide identity, with numerous differences located in CDRH3. None of the heavy chains would be considered clonally related to TG10, as defined as variable regions that use the same V and J germline genes, have the same length CDRH3, and have >70% nucleotide identity in the VDJ junction region. The most similar light chain had 96.8% identity at the nucleotide level.

### Example 2

This example shows that TG10 is highly selective for dPNAG and displays distinct binding relative to F598. Antibodies TG10 and F598 were recombinantly expressed and studied extensively using a glycan microarray. The microarray contained 816 different array components, including a wide range of N-linked and O-linked glycans, glycans from glycolipids, glycosaminoglycans, glycopeptides, and glycoproteins. In addition, the microarray included a set of 32 different PNAG-related pentasaccharides with varying numbers and position of acetyl groups.

Antibody selectivity and apparent K_{D} were determined using a NGP microarray. A pre-assay scan was performed to control for missing features. Selectivity and affinity of most mAbs were measured on the full array. A focused array was used to acquire an apparent K_{D} measurement. A slide module (8 well for full array and 16-well for focused array) was mounted onto each slide and blocking buffer (3% BSA in PBS, pH 7.4) was applied to each well of the array. Blocking was performed overnight at 4° C. mAbs were serial diluted in binding buffer (1% BSA in PBST, pH 7.4). Following the block, wells were washed 4× with wash buffer (PBST, PBS with 0.05% tween-20). Primary mAb incubation was performed for 2.5 hours at 37° C. The wells were washed 5× with wash buffer. A CY3-labeled secondary antibody specific for the Fc portion of human IgG was diluted 500 fold into binding buffer and added to each array well (Jackson ImmunoResearch, 109-165-098). Secondary mAb incubation was performed for 1.5 hours at 37° C. Wells were washed 5× with wash buffer. Slides were removed from the slide module and submerged in wash buffer for 5 minutes. Washed slides were dried by centrifugation @ 1000×g in a centrifuge quipped with a swing bucket rotor. Fluorescent scans were obtained at multiple PMT values to maximize dynamic range. Raw data was analyzed using GenePix 7.0 software as previously described. Selectivity profiles and apparent K_{D} curves were plotted in excel and Graphpad Prism. Apparent Kos were determined using one-site specific binding algorithm in Graphpad Prism.

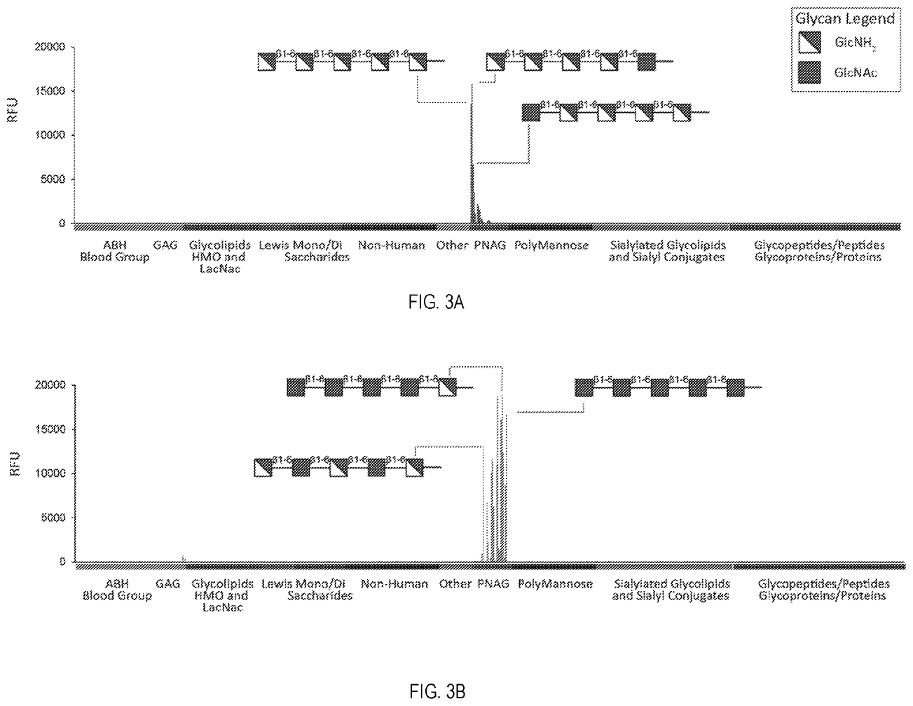

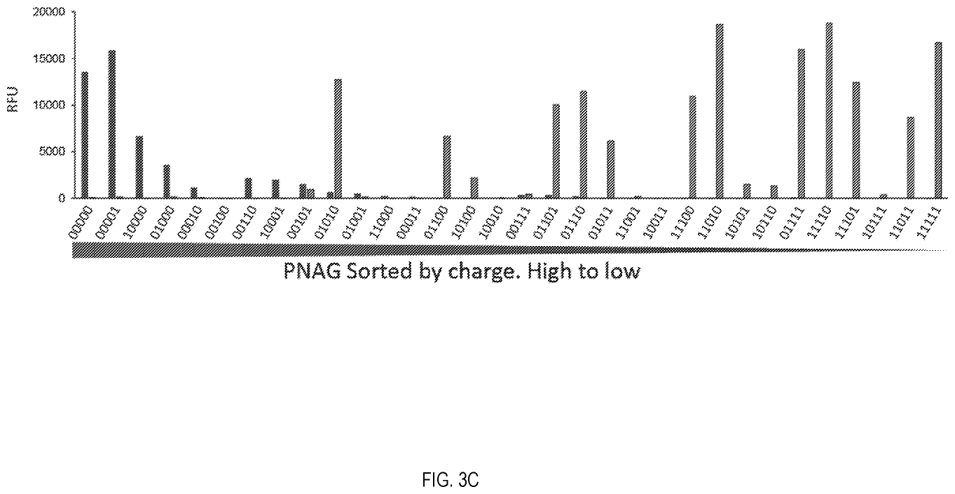

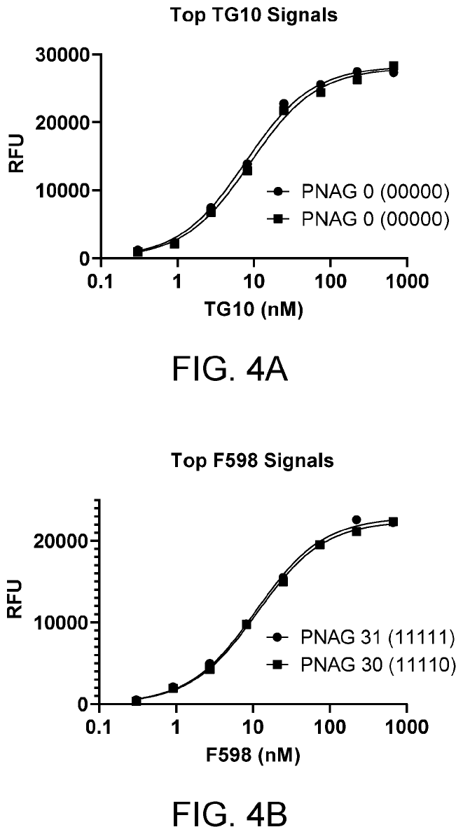

FIGS. 3A-3C depict array profiles of TG10 and F598. Signals in relative fluorescence units (RFUs) for TG10 on a 816-component array are shown. FIG. 3A shows a TG10 binding profile at 5 μg/ml. FIG. 3B shows a F598 binding profile at 5 μg/ml. FIG. 3C shows binding profiles of TG10 and F598 to the 32 PNAG-BSA conjugates on the array. PNAG conjugates are sorted by charge, or from highest degree of deacetylation to highest degree of acetylation. Glycan structures were created using GlycoGlyph. FIGS. 4A and 4B show binding curves and apparent Kos for the top two conjugates on the array. Signals are in relative fluorescence units (RFUs) for TG10 on a 816-component array. FIG. 4A shows TG10 binding with the highly deacetylated PNAG conjugates PNAG 0 and PNAG 1. FIG. 4B shows F598 binding with the highly N-acetylated PNAG conjugates PNAG 30 and PNAG 31.

As shown in FIGS. 3A and 3B, binding of both TG10 and F598 was constrained to the family of PNAG glycoconjugates present on the array. TG10 demonstrated excellent selectivity for the highly deacetylated PNAG conjugates PNAG 0 and PNAG 1 on the array (FIG. 4A), with apparent K_{D}s of 8.3 nM and 11.5 nM, respectively. PNAG 0 represents the glucosamine pentasaccharide conjugate. PNAG 1 contains glucosamine tetrasaccharide with an N-acetyl glucosamine at the reducing end. TG10 bound to PNAG 16, the glucosamine tetrasaccharide with an N-acetyl glucosamine at the terminal end with a slightly reduced apparent K_{D} of 19.0 nM. F598 showed zero binding to these heavily deacetylated PNAG glycoconjugates on the array. F598 preferentially bound to PNAG glycoconjugates with a high degree of acetylation in the pentasaccharide glycan. F598 bound to a wider range of glycoconjugates when compared to TG10, but the best recognition was to conjugates PNAG 31, PNAG 30, and PNAG 15, with apparent Kos of 11.0 nM, 11.5 nM, and 10.4 nM respectively (31 and 30 shown in FIG. 4B).

### Example 3

This example describes how key single point mutations HC W38A (SEQ ID NO: 47), HC D107A (SEQ ID NO: 41), HC D109K (SEQ ID NO: 48), LC Y38A (SEQ ID NO: 43), and LC D116A (SEQ ID NO: 49) produce functionally dead TG10 variants. A series of single point mutants were designed and synthesized to probe the binding site of TG10 with the goal of identifying residues that significantly influence selectivity and/or affinity. This information provides key insights for understanding recognition as well as enable engineering of variants that are inactive or have a different binding profile. 13 residues near or within the CDRs of the heavy and light chains were selected for evaluation (FIG. 2). Remarkably, several of these single point mutations resulted in complete or nearly complete loss of binding to the PNAG conjugates on the array.

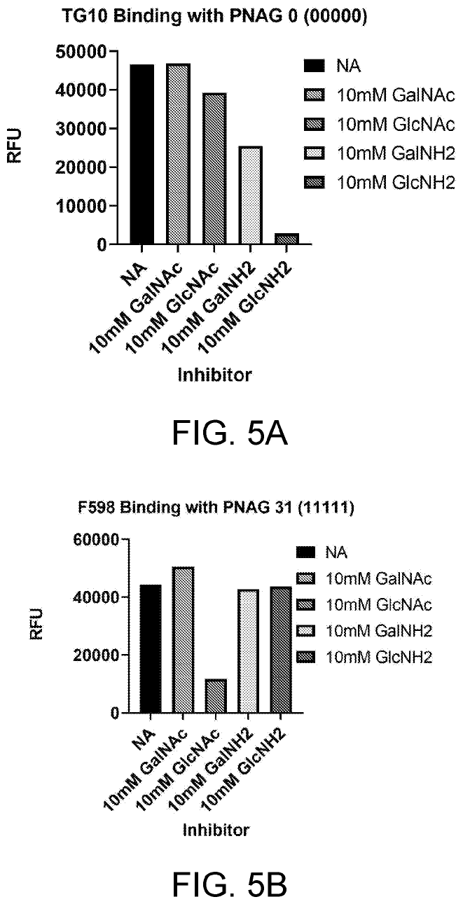

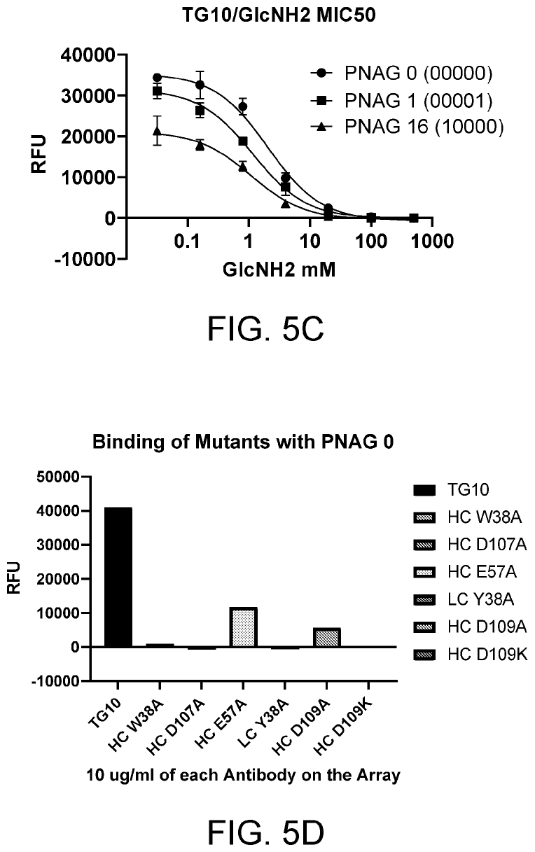

As shown in FIG. 5A, HC mutants W38A, D107A, D109K and LC mutants Y38A and D116A completely removed binding between TG10 and the fully deacetylated PNAG conjugate, PNAG 0. These key single point mutants also showed zero or very little binding with any of the array components at 100 μg/mL. HC mutants D109A and E57A were also shown to greatly reduce TG10/PNAG 0 binding interaction, but still retained some activity towards this array component. For comparison, TG10 displays an apparent K_{D} value for PNAG 0 of 8.3 nM (1.25 μg/mL) on the microarray; thus, a lack of any measurable binding at concentration of 100 μg/mL, about 65-fold above the apparent K_{D} value for TG10, signifies at least a 1000-fold decrease in affinity.

### Example 4

This example describes how TG10 binding is inhibited by glucosamine. For small molecule inhibition assays, arrays were blocked and washed as previously described. Antibodies F598 and TG10 were diluted to 5 μg/mL in binding buffer. 20 mM N-Acetyl glucosamine and 20 mM glucosamine in binding buffer was prepared and an equal volume of the monosaccharide solutions were added to the diluted antibodies. Antibody/monosaccharide samples were incubated at room temperature for 30 min with rotation prior to being applied to the array. For the TG10 glucosamine IC50 inhibition assay, TG10 was diluted to 30 nM in binding buffer. A pH adjusted 1M stock of Glucosamine.HCl in PBS with 3% BSA (pH 7.2) was prepared and a serial dilution in binding buffer was made. Equal volumes of TG10 and each of the serial diluted glucosamine stocks was combined. Antibody/monosaccharide samples were incubated at RT for 30 min with rotation prior to being applied to the array.

FIGS. 5A-5D show antibody binding inhibited by monosaccharides and eliminated by multiple single point mutants. FIG. 5A shows that TG10 binding to the fully deacetylated PNAG 0 is inhibited by 10 mM GlcNH2>GalNH2>>>GlcNAc or GalNAc. FIG. 5B shows that F598 binding to the fully N-acetylated PNAG31 is inhibited by 10 mM GlcNAc>>GlcNH2, GalNH2 or GlcNAc. FIG. 5C shows that GlcNH2 IC50 values for TG10 and the dPNAG array components PNAG 0, PNAG 1, and PNAG 16. FIG. 5D shows that single point mutations eliminated TG10 binding with the fully deacetylated array component PNAG 0. Signals in relative fluorescence units (RFUs) for TG10 on an 816-component array.

Monosaccharide inhibition of both TG10 and F598 was studied on the array. The antibodies were incubated with 10 mM glucosamine, galactosamine, N-acetyl glucosamine, and N-acetyl galactosamine and then applied to the array. The binding of TG10 with PNAG 0 and of F598 with PNAG 31 were analyzed with and without monosaccharide inhibitors (FIGS. 5A-5D). Glucosamine had the greatest effect on TG10 binding to PNAG 0, reducing signal intensity by 94% at 10 mM GalNH2 (100% at 100 mM GalNH2). As expected, N-acetyl glucosamine inhibited F598 binding with PNAG 31, reducing RFU by 74% at 10 mM GlcNAc and 99% at 100 mM GlcNAc. TG10 binding was unchanged by 10 mM N-acetyl galactosamine but was slightly inhibited by both N-acetyl glucosamine (15%) and galactosamine (45%), whereas F598 binding was not inhibited by monosaccharides other than N-acetyl glucosamine. To further illuminate the small molecule inhibition properties of glucosamine on TG10, IC50 values for glucosamine was determined by titrating GlcNH2 with TG10. The IC50 of glucosamine was found to be around 1-2 mM for the three best binding conjugates on the array (FIG. 5C).

### Example 5

This example demonstrates that TG10 binds to a distinct S. epidermidis population within biofilm compared to F598. For antibody labeling, recombinantly expressed antibodies were labeled with fluorescent dyes using Invitrogen antibody dye labeling kits. Concentration and labeling efficiency was confirmed using nanodrop. For S. epidermidis biofilm formation and confocal microscopy, Staphylococcus epidermidis (Winslow and Winslow) Evans ATCC 14990 was resuspended and cultured on tryptic soy broth agar plates according to ATCC recommended procedures. Single colonies were picked into 5 mL Tryptic Soy Broth (TSB) supplemented with 0.25% glucose (BD™ TSB) and bacteria were cultured overnight at 37 C with shaking (200 RPM). The overnight culture was diluted 1:100 into aerated TSB26. 400 μl (microliters) of the aerated culture was pipetted into 8-well chambered coverglass lab-tek II system suitable for confocal microscopy. Biofilms were allowed to mature for 20 hrs @37C without agitation. Mature films were washed 2×500 μl PBS to remove planktonic bacteria. The films were fixed in 4% paraformaldehyde in PBS for 30 minutes, washed 2×500 μl PBS and stained with fluorescently labeled antibodies diluted to 5 μg/ml. The labeled films were then washed 2×500 μl PBS and then stained for 10 minutes with Hoechst stain @ 10 μg/ml. The films were washed a final time and kept under PBS for imaging. Stained biofilms fixed on chambered coverglass were imaged using a Leica TCS SP8× confocal microscope equipped with a 63× oil immersion objective lens. Settings are pinhole and laser power. Z Stacks at were taken at 1200×1200 resolution. Stacks were rendered in Leica LAS X software.

Given the specific and unique binding profile of TG10 on the array and the array-directed method of discovery, finding natural targets for the dPNAG-binding antibody was a significant and challenging endeavor. Guided primarily from experiments on F598, the search for a natural target for TG10 was begun by profiling cultures of 5 suspected PNAG producing microorganisms: Mycobacterium smegmatis (ATCC® 700084™), Staphylococcus epidermidis (ATCC® 35984™), Aspergillus flavus (ATCC® 200026™), Escherichia coli (Migula) (ATCC® 700926™), and Candida albicans (ATCC® 14053). In an initial experiment, Alexa Fluor 488 labeled TG10 was incubated with suspension cultures of the 5 microorganisms. Gratifyingly, after spinning down the culture of S. epidermidis, the pelleted sample was visibly green and fluorescent microscopy confirmed a strong fluorescent signal in the spun-down S. epidermidis culture (data not shown). With this positive evidence, S. epidermidis biofilms became an early frontrunner as a natural target for TG10.

Efforts were focused on high-resolution imaging of cultured S. epidermidis biofilms. Briefly, overnight S. epidermidis suspension cultures were diluted into aerated TSB and plated into chambered coverglass slides. Following overnight biofilm development, planktonic cells were washed away, and the biofilm was incubated with fluorescently labeled TG10 and F598. In parallel to the different array profiles of TG10 and F598, the two antibodies consistently localized to different populations within the biofilm.

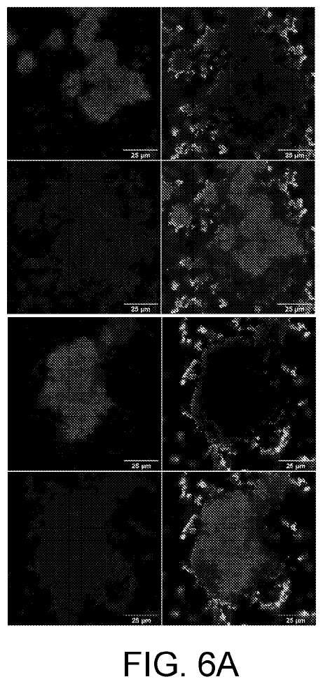

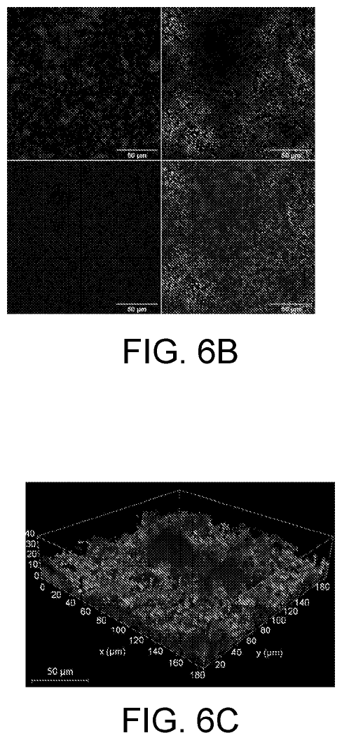

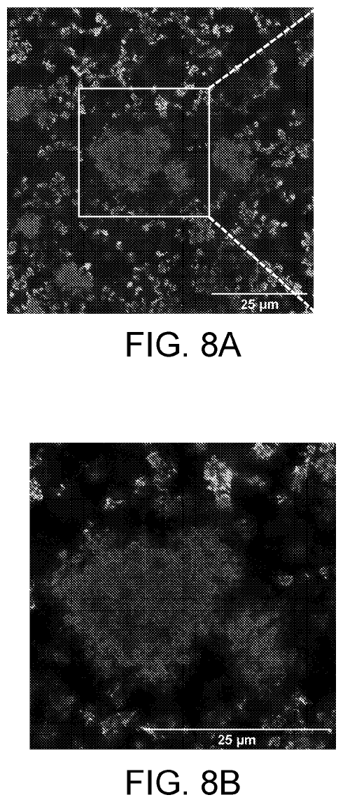

FIGS. 6A-6C, S. show epidermidis biofilms stained with monoclonal antibodies TG10 and F598. TG10-647, F598-488, and DNA stained with Hoechst 33342. In FIGS. 6A and 6B, three channels are split with the overlay in the bottom right. FIG. 6A shows a representative microcolony within the biofilm stains strongly with TG10. F598 staining is strongest on the perimeter of the microcolonies. FIG. 6B shows representative clusters of TG10 positive cells surrounds by F598 staining. FIG. 6C is a 3D rendering of Z-stack images. FIG. 8A shows a confocal image of TG10 and F598. TG10-647, F598-488, and DNA stained with Hoechst 33342 differentially stained. Zoomed inset of microcolony shows individual cells of the microcolony.

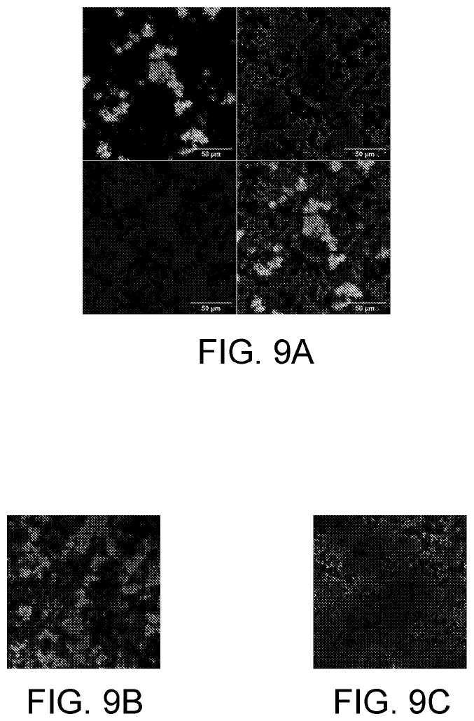

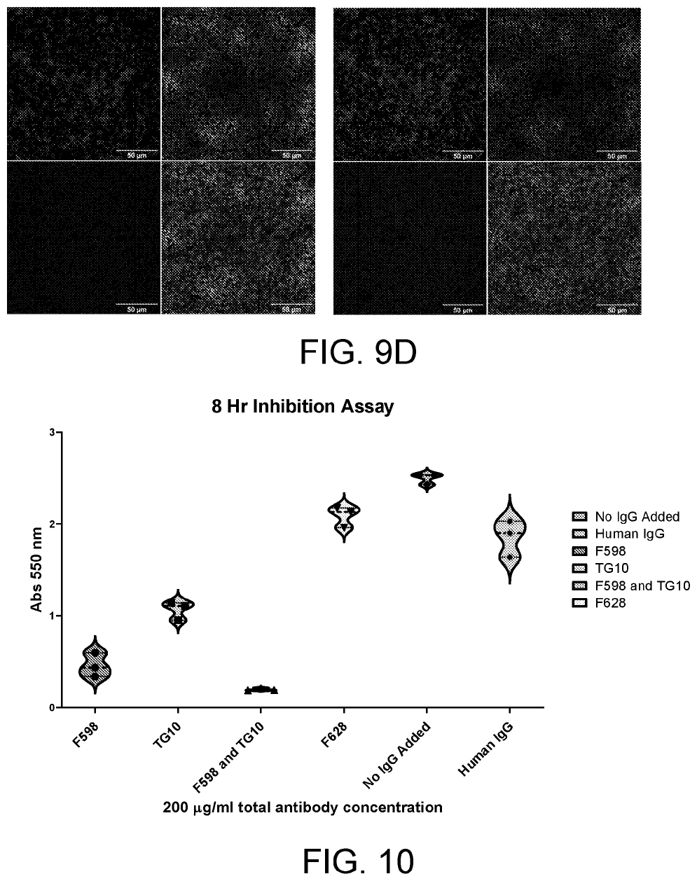

As seen in FIG. 6A, an Alexa Fluor 647 labeled TG10 isolated to core cells within the microcolonies distributed throughout the biofilm. In contrast, an Alexa fluor 488 labeled F598 bound to the cells on the periphery of the microcolonies. As shown in the three-color overlayed confocal image in FIGS. 6A, 6B, and 8A, individual cells within the biofilm microcolony core can be identified with the 647 labeled TG10. Remarkably, TG10 and F598 share very little colocalization, both occupying unique environments within the biofilm matrix. A wider 2D view of the biofilm (FIG. 6B), shows a general trend of dense TG10 binding clusters of cells which are surrounded by a diffuse web of F598 binding material and cells. Another intriguing characteristic observed with TG10 and F598 biofilm binding is the enhanced localization of TG10 to the basement cells attached to the slide surface when compared to F598. As shown in FIG. 9C, the basement of the biofilm is strongly stained with TG10 when compared to F598. The 3D architecture of the biofilm is observed in Z stack shown in FIG. 6C. TG10 localizes on the basement of the biofilm and predominates within the microcolonies growing within and protruding from the biofilm. On the other hand, F598 binds to the surfaces of the TG10 associated microcolonies and accumulates on cells and matrix at the surface of the biofilm.

FIGS. 9A-9D show confocal Images of TG10 and F598. FIG. 9A shows TG10-488, F598-647, and DNA stained with Hoechst 33342 differentially stained. FIG. 9B shows TG10-488. FIG. 9C shows F598-488 and DNA stained with Hoechst 33342. FIG. 9D shows basement, or lower areas of the biofilm show elevated TG10 staining relative to F598. To ensure that fluorescent labels were not playing a role in antibody penetration into the biofilms, fluorophore labels were swapped, and the assay was repeated. As visible in FIG. 9A, a 647 labeled F598 again bound to cells surrounding the 488-labeled TG10 binding microcolony core. Biofilms were stained individually with 488-labeled TG10 or 488-labeled F598, shown in FIGS. 9B and 9C, respectively, confirming the localization of TG10 to cells at the core of the biofilm's microcolonies versus F598 binding the outer layer of cells.

### Example 6

This example demonstrates that TG10 binds to planktonic S. aureus strains ATCC 29213 and Xen 36. TG10 binding was evaluated in the PNAG producing strains of S. aureus ATCC 29213 and Xen 36 following overnight culturing with shaking in TSB and TB, respectively. The cells were pelleted, fixed in 4% paraformaldehyde and coated on glass chips. The plated cells were extensively washed followed by incubation with 488-labeled TG10 or 488-labeled F598 prior fluorescent microscopy. For evaluating TG10 and F598 binding with S. aureus, S. aureus ATCC 29212 and Xen 36 were grown in TSB and LB overnight, respectively. Cells were pelleted and fixed in 4% paraformaldehyde. Cells were calibrated to OD 0.5. 100 μl suspension were used for each sample. Each sample were pelleted and resuspended in 20 μg/mL mAb TG10, F598 or both. After shaking for 2 hours under ambient temperature, cells were pelleted, washed and transferred to glass chips and allowed to air dry. A cover slide was mounted via anti-fade media.

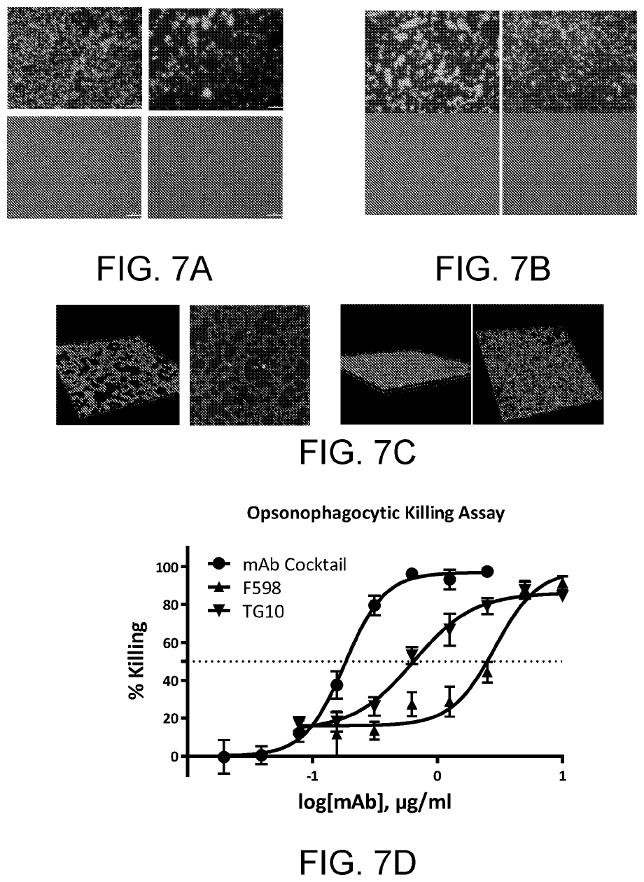

FIGS. 7A-7D show antibody binding to S. aureus and activity in an opsonophagocytic killing (OPK) assay. FIG. 7A depicts fluorescence microscopy (top) and brightfield images (bottom) of FITC labeled TG10 binding to ATCC 29213 and Xen 36 cells (scale bar: 10 μm). FIG. 7B depicts fluorescence microscopy (top) and brightfield images (bottom) of FITC labeled F598 binding to ATCC 29213 and Xen 36 cells (scale bar: 10 μm). FIG. 7C depicts confocal images of FITC-labeled F598 and 647-labeled TG10 binding to ATCC 29213 and Xen 36 cells (scale bar: 2 μm). FIG. 7D shows results graphed of an OPK assay of TG10, F598 and mAb cocktail. Killing efficiency was fitted to log total final concentration of mAbs via 4-parameter logistic method. EC50 was calculated based on the concentration to give 50% killing.

TG10 bound to ATCC 29213 and Xen 36 cells, shown in FIG. 7A. Likewise, F598 was observed to bind to both strains of S. aureus (FIG. 7B). To determine if there were any populations within the planktonic suspensions that preferentially bound TG10, TG10 and F598 binding was evaluated using confocal microscopy. 647-labeled TG10 and a FITC-labeled F598 were co-incubated with the S. aureus strains at a concentration of 20 μg/ml. Interestingly, while not all cells were strongly stained with TG10 when co-incubated with F598, a distinct population of cells in each strain preferentially bound to TG10 over F598 (FIG. 7C).

### Example 7

This example demonstrates that TG10 kills S. aureus Xen 36 cells and enhances F598 activity in an OPK assay. The opsonic killing activity of antibodies TG10 and F598 were studied individually and as a cocktail of the two mAbs. The antibodies were serially diluted and preincubated with Xen 36 cells for 30 minutes at 37° C. Following the addition of rabbit complement, HI-60 cells were added and bioluminescence was measured after 2 hours. Killing was calculated by the following equation: (RLU(Max)−RLU(mAb))/(RLU(Max)−RLU(blank))*100%. Killing efficiency was fitted to log total final concentration of mAbs via 4-parameter logistic method and shown in FIG. 7D. EC50 was calculated based on the concentration to give 50% killing. EC50s were calculated for F598, TG10, and the mAb cocktail to be 2.50, 0.65, and 0.19 g/ml, respectively. TG10 outperformed F598 in this OPK assay. The cocktail of the two mAbs showed an additive or synergistic response when compared to the individual monoclonal antibodies. Providing additional evidence that there are different subpopulations within the cultures that display PNAGs with vastly different degrees of acetylation which can be recognized by the unique CDR of TG10.

For the opsonic killing assay, 10⁵ cfu S. aureus Xen 36 cells were suspended in 20 mL RPMI-1640. Serial diluted mAb were added in 5 mL PBS. After incubation in 37° C. for 30 mins, 25 mL Xen 36-absorbed rabbit complement was added. 10⁵ induced HL-60 cells (1% DMF, 7 days) were added in 50 μl RPMI-1640. Bioluminescence was recorded at 2 hours. mAb omitted group was used as blank. Bleach was added at 2 hours as positive control. Normalized killing was calculated by [RLU(blank)−RLU(mAb)]/[RLU(blank)−RLU(bleach)]. Killing efficiency was fitted to the final concentration of mAbs via 4-parameter logistic method. EC50 was calculated based on the concentration needed to kill 50% bacterial cells compared to control. FIG. 7D depicts antibody binding to S. aureus and activity in OPK assay. Opsonophagocytic killing (OPK) assay of TG10, F598 and mAb cocktail are shown. Killing efficiency was fitted to log total final concentration of mAbs via 4-parameter logistic method. EC50 was calculated based on the concentration to give 50% killing.

### Example 8

This example demonstrates that TG10 inhibits S. epidermidis biofilm formation similar to F598, and a mAb cocktail shows enhanced inhibition. TG10 and F598 were studied to evaluate their inhibitory effects on biofilm formation in an in vitro S. epidermidis biofilm formation inhibition assay. In addition to TG10, F598, and a cocktail of the two mAbs, human IgG and a PNAG-binding mAb discovered alongside F598 were included as controls. An overnight culture of S. epidermidis was diluted 1:100 in aerated TSB. Antibodies were serial diluted in TSB and added to the aerated S. epidermidis culture. Cells were incubated with the antibodies at room temperature for 30 minutes and then plated on 96-well tissue culture plates. After 8 hours at 37° C., the plates were washed with water and stained with a 0.1% solution of crystal violet. After being thoroughly washed and dried, the crystal violet stained biofilms were dissolved in 30% acetic acid, transferred to a fresh ELISA plate and the 550 nm absorbance was measured in a plate reader.

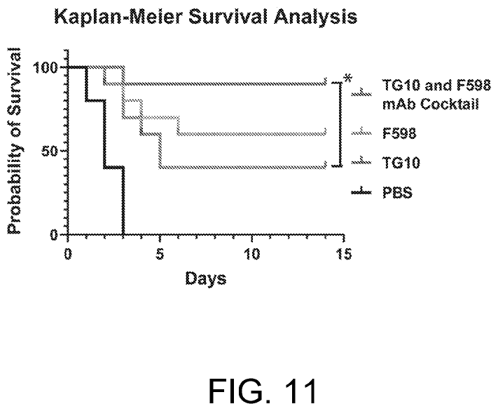

FIG. 10 shows a violin plot of S. epidermidis biofilm growth inhibition. Antibodies and controls were incubated at 200 μg/mL for 8 hours, washed, and stained with crystal violet. UV absorbance at 550 nm was used to quantify crystal violet binding to PNAG. As shown in FIG. 10, 200 μg/mL of either F598 or TG10 was sufficient to inhibit >50% percent of biofilm formation at the 8-hour timepoint relative to no IgG or the isotype control F628. When TG10 and F598 were combined to a final concentration of 100 μg/mL of each antibody, the near complete inhibition of growth was observed.

### Example 9

This example demonstrates that immunization of mice with TG10 can increase survival of mice subsequently challenged with a lethal dosage of Staphylococcus aureus. An in vivo study was performed to evaluate the prophylactic efficacy of TG10 against a S. aureus lethal dose challenge in a murine model of infection. TG10 and F598 were evaluated as monotherapies and as a mAb cocktail. The results of this initial mouse model show that TG10 and F598 produced increased survival in the treated group when compared to the PBS control group (FIG. 11). Significantly, when TG10 and F598 were co-administered as a mAb cocktail, a synergistic response was observed compared to the monotherapy arm of the study. The combination provided 90% survival.

Kaplan-Meier survival analysis shows effective passive protection in the mouse model. Balb/C mice aged 12 weeks (n=10/group) were passively immunized with 200 μg F598, 200μ g TG10, or 100μ g F598+100μ g TG10 in 100 μl PBS intraperitoneally. 100 μl PBS was injected intraperitoneally in the negative control group (n=5). One hour later, 200 million cfu S. aureus ATCC 29213 cells were administrated via tail vein. Mice condition was monitored. A mouse unable to move or which lost more than 20% body mass was euthanized. Survival was better in all three groups when compared to the PBS control arm. Mouse survival when passively immunized with TG10 and F598 was significantly improved compared to TG10 passive immunization alone. Results are shown in FIG. 11.

The present disclosure includes the following aspects/embodiments/features in any order and/or in any combination:
1. An antibody or antigen-binding fragment thereof comprising a heavy chain variable region and a light chain variable region, the heavy chain variable region having an amino acid sequence comprising SEQ ID NO: 3, and the light chain variable region having an amino acid sequence comprising SEQ ID NO: 12.
2. An antibody or antigen-binding fragment thereof comprising a heavy chain variable region and a light chain variable region, the heavy chain variable region having an amino acid sequence comprising SEQ ID NO: 3, and the light chain variable region having an amino acid sequence comprising SEQ ID NO: 24.
3. An antibody comprising a heavy chain variable region and a light chain variable region,
   wherein the heavy chain variable region comprises an amino acid sequence selected from SEQ ID NO: 3, 31, 34, 36-39, 41, and 42; and
   wherein the light chain variable region comprises an amino acid sequence selected from SEQ ID NOS: 12, 24, 40, and 43.
4. An antibody or antigen-binding fragment thereof comprising a heavy chain variable region and a light chain variable region,
   wherein the heavy chain variable region comprises an amino acid sequence selected from SEQ ID NOS: 3, 31, 34, 36-39, 41, and 42; and
   wherein the light chain variable region having an amino acid sequence comprising SEQ ID NO: 12
5. An antibody or antigen-binding fragment thereof comprising a heavy chain variable region and a light chain variable region,
   wherein the heavy chain variable region has an amino acid sequence comprising SEQ ID NO: 3; and
   wherein the light chain variable region comprises an amino acid sequence selected from SEQ ID NOS: 12, 30, 40, and 43.
6. An antibody or antigen-binding fragment thereof comprising a heavy chain variable region and a light chain variable region, the heavy chain variable region having an amino acid sequence comprising an amino acid sequence at least 92% identical to SEQ ID NO: 3, and the light chain variable region having an amino acid sequence comprising an amino acid sequence at least 92% identical to SEQ ID NO: 12, wherein the antibody or antigen-binding fragment thereof binds to deacetylated poly-N-acetyl glucosamine (dPNAG) and fewer than about 50% of the beta-1,6-linked glucosamine residues are N-acetylated, or binds to a saccharide comprising beta-1,6-linked glucosamine residues, the saccharide comprising at least five sugar residues, or binds to both.
7. An antibody or antigen-binding fragment thereof comprising a heavy chain variable region and a light chain variable region, the heavy chain variable region having an amino acid sequence comprising an amino acid sequence at least 95% identical to SEQ ID NO: 3, and the light chain variable region having an amino acid sequence comprising an amino acid sequence at least 95% identical to SEQ ID NO: 12, wherein the antibody or antigen-binding fragment thereof binds to deacetylated poly-N-acetyl glucosamine (dPNAG) and fewer than about 50% of the beta-1,6-linked glucosamine residues are N-acetylated, or binds to a saccharide comprising beta-1,6-linked glucosamine residues, the saccharide comprising at least five sugar residues, or binds to both.
8. An antibody or antigen-binding fragment thereof comprising a heavy chain variable region and a light chain variable region,
   wherein the heavy chain variable region comprises an amino acid sequence at least 92% identical to an amino acid sequence selected from SEQ ID NO: 3,
   wherein the light chain variable region comprises an amino acid sequence at least 92% identical to an amino acid sequence selected from SEQ ID NO: 24, and
   wherein the antibody or antigen-binding fragment thereof binds to deacetylated poly-N-acetyl glucosamine (dPNAG) and fewer than about 25% of the beta-1,6-linked glucosamine residues are N-acetylated, or binds to a saccharide comprising beta-1,6-linked glucosamine residues, the saccharide comprising at least five sugar residues, or binds to both.
9. An antibody or antigen-binding fragment thereof comprising a heavy chain variable region and a light chain variable region,
   wherein the heavy chain variable region comprises an amino acid sequence at least 98% identical to an amino acid sequence selected from SEQ ID NO: 3,
   wherein the light chain variable region comprises an amino acid sequence at least 98% identical to an amino acid sequence selected from SEQ ID NO: 24, and
   wherein the antibody or antigen-binding fragment thereof binds to deacetylated poly-N-acetyl glucosamine (dPNAG) and fewer than about 25% of the beta-1,6-linked glucosamine residues are N-acetylated, or binds to a saccharide comprising beta-1,6-linked glucosamine residues, the saccharide comprising at least five sugar residues, or binds to both.
10. An antibody or antigen-binding fragment thereof comprising:
   a heavy chain variable region comprising a CDRH1 sequence comprising SEQ ID NO: 4, a CDRH2 sequence comprising SEQ ID NO: 6, or a CDRH3 sequence comprising SEQ ID NO: 7, or any combination thereof;
   a light chain variable region comprising a CDRL1 sequence comprising SEQ ID NO: 13, a CDRL2 sequence comprising the amino acid sequence GAS, or a CDRL3 sequence comprising SEQ ID NO: 16, or any combination thereof,
   wherein the antibody or antigen-binding fragment thereof binds to deacetylated poly-N-acetyl glucosamine (dPNAG) and fewer than about 50% of the beta-1,6-linked glucosamine residues are N-acetylated, or binds to a saccharide comprising beta-1,6-linked glucosamine residues, the saccharide comprising at least five sugar residues, or binds to both.
11. An antibody or antigen-binding fragment thereof comprising:
   a heavy chain variable region comprising two or more of a CDRH1 sequence comprising SEQ ID NO: 4, a CDRH2 sequence comprising SEQ ID NO: 6, and a CDRH3 sequence comprising SEQ ID NO: 7,
   wherein the antibody or antigen-binding fragment thereof binds to deacetylated poly-N-acetyl glucosamine (dPNAG) and fewer than about 50% of the beta-1,6-linked glucosamine residues are N-acetylated, or binds to a saccharide comprising beta-1,6-linked glucosamine residues, the saccharide comprising at least five sugar residues, or binds to both.
12. An antibody or antigen-binding fragment thereof comprising:
   a light chain variable region comprising two or more of a CDRL1 sequence comprising SEQ ID NO: 13, a CDRL2 sequence comprising the amino acid sequence GAS, and a CDRL3 sequence comprising SEQ ID NO: 16,
   wherein the antibody or antigen-binding fragment thereof binds to deacetylated poly-N-acetyl glucosamine (dPNAG) and fewer than about 50% of the beta-1,6-linked glucosamine residues are N-acetylated, or binds to a saccharide comprising beta-1,6-linked glucosamine residues, the saccharide comprising at least five sugar residues, or binds to both.
13. An antibody or antigen-binding fragment thereof comprising:
   a heavy chain variable region comprising two or more of a CDRH1 sequence comprising SEQ ID NO: 4, a CDRH2 sequence comprising SEQ ID NO: 6, and a CDRH3 sequence comprising SEQ ID NO: 7; and
   a light chain variable region comprising two or more of a CDRL1 sequence comprising SEQ ID NO: 13, a CDRL2 sequence comprising the amino acid sequence GAS, and a CDRL3 sequence comprising SEQ ID NO: 16,
   wherein the antibody or antigen-binding fragment thereof binds to deacetylated poly-N-acetyl glucosamine (dPNAG) and fewer than about 50% of the beta-1,6-linked glucosamine residues are N-acetylated, or binds to a saccharide comprising beta-1,6-linked glucosamine residues, the saccharide comprising at least five sugar residues, or binds to both.
14. An antibody or antigen-binding fragment thereof comprising:
   a heavy chain variable region comprising a CDRH1 sequence comprising SEQ ID NO: 4, a CDRH2 sequence comprising SEQ ID NO: 6, and a CDRH3 sequence comprising SEQ ID NO: 7,
   wherein the antibody or antigen-binding fragment thereof binds to deacetylated poly-N-acetyl glucosamine (dPNAG) and fewer than about 50% of the beta-1,6-linked glucosamine residues are N-acetylated, or binds to a saccharide comprising beta-1,6-linked glucosamine residues, the saccharide comprising at least five sugar residues, or binds to both.
15. An antibody or antigen-binding fragment thereof comprising:
   a light chain variable region comprising a CDRL1 sequence comprising SEQ ID NO: 13, a CDRL2 sequence comprising the amino acid sequence GAS, and a CDRL3 sequence comprising SEQ ID NO: 16,
   wherein the antibody or antigen-binding fragment thereof binds to deacetylated poly-N-acetyl glucosamine (dPNAG) and fewer than about 50% of the beta-1,6-linked glucosamine residues are N-acetylated, or binds to a saccharide comprising beta-1,6-linked glucosamine residues, the saccharide comprising at least five sugar residues, or binds to both.
16. An antibody or antigen-binding fragment thereof comprising:
   a heavy chain variable region comprising a CDRH1 sequence comprising SEQ ID NO: 4, a CDRH2 sequence comprising SEQ ID NO: 6, and a CDRH3 sequence comprising SEQ ID NO: 7; and
   a light chain variable region comprising a CDRL1 sequence comprising SEQ ID NO: 13, a CDRL2 sequence comprising the amino acid sequence GAS, and a CDRL3 sequence comprising SEQ ID NO: 16,
   wherein the antibody or antigen-binding fragment thereof binds to deacetylated poly-N-acetyl glucosamine (dPNAG) and fewer than about 50% of the beta-1,6-linked glucosamine residues are N-acetylated, or binds to a saccharide comprising beta-1,6-linked glucosamine residues, the saccharide comprising at least five sugar residues, or binds to both.
17. An antibody or antigen-binding fragment thereof comprising:
   a heavy chain variable region comprising a CDRH1 sequence comprising SEQ ID NO: 4, a CDRH2 sequence comprising SEQ ID NO: 6, or a CDRH3 sequence comprising SEQ ID NO: 7, or any combination thereof;
   a light chain variable region comprising a CDRL1 sequence comprising SEQ ID NO: 25, a CDRL2 sequence comprising the amino acid sequence GAS, or a CDRL3 sequence comprising SEQ ID NO: 27, or any combination thereof,
   wherein the antibody or antigen-binding fragment thereof binds to deacetylated poly-N-acetyl glucosamine (dPNAG) and fewer than about 25% of the beta-1,6-linked glucosamine residues are N-acetylated, or binds to a saccharide comprising beta-1,6-linked glucosamine residues, the saccharide comprising at least five sugar residues, or binds to both.
18. The antibody or antigen-binding fragment thereof of any preceding or following embodiment/feature/aspect, wherein the heavy chain variable region further comprises a variable region heavy chain framework 1 (HC-FR1) sequence of SEQ ID NO: 8, a HC-FR2 sequence of SEQ ID NO: 9, a HC-FR3 sequence of SEQ ID NO: 10, or a HC-FR4 sequence of SEQ ID NO: 11, or any combination thereof.
19. The antibody or antigen-binding fragment thereof of any preceding or following embodiment/feature/aspect, wherein the light chain variable region further comprise a variable region light chain framework 1 (LC-FR1) sequence of SEQ ID NO: 18, an LC-FR2 sequence of SEQ ID NO: 19, an LC-FR3 sequence of SEQ ID NO: 20; or an LC-FR4 sequence of SEQ ID NO: 21, or any combination thereof.
20. The antibody or antigen-binding fragment thereof of any preceding or following embodiment/feature/aspect,
   wherein the heavy chain variable region further comprises a variable region heavy chain framework 1 (HC-FR1) sequence of SEQ ID NO: 8, a HC-FR2 sequence of SEQ ID NO: 9, a HC-FR3 sequence of SEQ ID NO: 10, or a HC-FR4 sequence of SEQ ID NO: 11, or any combination thereof; and
   wherein the light chain variable region further comprises a variable region light chain framework 1 (LC-FR1) sequence of SEQ ID NO: 18, an LC-FR2 sequence of SEQ ID NO: 19, an LC-FR3 sequence of SEQ ID NO: 20; or an LC-FR4 sequence of SEQ ID NO: 21, or any combination thereof.
21. The antibody or antigen-binding fragment thereof of any preceding or following embodiment/feature/aspect,
   wherein the heavy chain variable region further comprises two or more a variable region heavy chain framework 1 (HC-FR1) sequence of SEQ ID NO: 8, a HC-FR2 sequence of SEQ ID NO: 9, a HC-FR3 sequence of SEQ ID NO: 10, and a HC-FR4 sequence of SEQ ID NO: 11; and
   wherein the light chain variable region further comprises two or more of a variable region light chain framework 1 (LC-FR1) sequence of SEQ ID NO: 18, an LC-FR2 sequence of SEQ ID NO: 19, an LC-FR3 sequence of SEQ ID NO: 20; and an LC-FR4 sequence of SEQ ID NO: 21.
22. The antibody or antigen-binding fragment thereof of any preceding or following embodiment/feature/aspect, wherein the heavy chain variable region further comprises a variable region heavy chain framework 1 (HC-FR1) sequence of SEQ ID NO: 8, a HC-FR2 sequence of SEQ ID NO: 9, a HC-FR3 sequence of SEQ ID NO: 10, and a HC-FR4 sequence of SEQ ID NO: 11.
23. The antibody or antigen-binding fragment thereof of any preceding or following embodiment/feature/aspect, wherein the light chain variable region further comprises a variable region light chain framework 1 (LC-FR1) sequence of SEQ ID NO: 18, an LC-FR2 sequence of SEQ ID NO: 19, an LC-FR3 sequence of SEQ ID NO: 20; and an LC-FR4 sequence of SEQ ID NO: 21.
24. The antibody or antigen-binding fragment thereof of any preceding or following embodiment/feature/aspect, wherein the light chain variable region further comprise a variable region light chain framework 1 (LC-FR1) sequence of SEQ ID NO: 28, an LC-FR2 sequence of SEQ ID NO: 29, an LC-FR3 sequence of SEQ ID NO: 30; or an LC-FR4 sequence of SEQ ID NO: 21, or any combination thereof.
25. The antibody or antigen-binding fragment thereof of any preceding or following embodiment/feature/aspect,
   wherein the heavy chain variable region further comprises a variable region heavy chain framework 1 (HC-FR1) sequence of SEQ ID NO: 8, a HC-FR2 sequence of SEQ ID NO: 9, a HC-FR3 sequence of SEQ ID NO: 10, or a HC-FR4 sequence of SEQ ID NO: 11, or any combination thereof; and
   wherein the light chain variable region further comprises a variable region light chain framework 1 (LC-FR1) sequence of SEQ ID NO: 28, an LC-FR2 sequence of SEQ ID NO: 29, an LC-FR3 sequence of SEQ ID NO: 30; or an LC-FR4 sequence of SEQ ID NO: 21 or any combination thereof.
26. The antibody or antigen-binding fragment thereof of any preceding or following embodiment/feature/aspect,
   wherein the heavy chain variable region further comprises two or more a variable region heavy chain framework 1 (HC-FR1) sequence of SEQ ID NO: 8, a HC-FR2 sequence of SEQ ID NO: 9, a HC-FR3 sequence of SEQ ID NO: 10, and a HC-FR4 sequence of SEQ ID NO: 11; and
   wherein the light chain variable region further comprises two or more of a variable region light chain framework 1 (LC-FR1) sequence of SEQ ID NO: 28, an LC-FR2 sequence of SEQ ID NO: 29, an LC-FR3 sequence of SEQ ID NO: 30; and an LC-FR4 sequence of SEQ ID NO: 31.
27. An antibody or antigen-binding fragment thereof comprising a heavy chain variable region comprising an amino acid sequence selected of SEQ ID NO: 3, wherein the antibody or antigen-binding fragment thereof binds to deacetylated poly-N-acetyl glucosamine (dPNAG) and fewer than about 50% of the beta-1,6-linked glucosamine residues are N-acetylated, or binds to a saccharide comprising beta-1,6-linked glucosamine residues, the saccharide comprising at least five sugar residues, or binds to both.
28. An antibody or antigen-binding fragment thereof comprising a light chain variable region comprising an amino acid sequence of SEQ ID NO: 12, wherein the antibody or antigen-binding fragment thereof binds to deacetylated poly-N-acetyl glucosamine (dPNAG) and fewer than about 50% of the beta-1,6-linked glucosamine residues are N-acetylated, or binds to a saccharide comprising beta-1,6-linked glucosamine residues, the saccharide comprising at least five sugar residues, or binds to both.
29. An antibody or antigen-binding fragment thereof comprising a light chain variable region comprising an amino acid sequence of SEQ ID NO: 24, wherein the antibody or antigen-binding fragment thereof binds to deacetylated poly-N-acetyl glucosamine (dPNAG) and fewer than about 25% of the beta-1,6-linked glucosamine residues are N-acetylated, or binds to a saccharide comprising beta-1,6-linked glucosamine residues, the saccharide comprising at least five sugar residues, or binds to both.
30. The antibody or antigen-binding fragment thereof of any preceding or following embodiment/feature/aspect, wherein the antibody or antigen-binding fragment thereof binds to deacetylated beta-1,6-poly-N-acetyl glucosamine that is at least 10% deacetylated, or binds to a saccharide comprising beta-1,6-linked glucosamine residues, the saccharide comprising at least five sugar residues, or binds to both.
31. The antibody or antigen-binding fragment thereof of any preceding or following embodiment/feature/aspect, wherein the antibody or antigen-binding fragment thereof binds to deacetylated beta-1,6-poly-N-acetyl glucosamine that is at least 50% deacetylated, or binds to a saccharide comprising beta-1,6-linked glucosamine residues, the saccharide comprising at least five sugar residues, or binds to both.
32. The antibody or antigen-binding fragment thereof of any preceding or following embodiment/feature/aspect, wherein the antibody or antigen-binding fragment thereof binds to deacetylated beta-1,6-poly-N-acetyl glucosamine that is at least 75% deacetylated, or binds to a saccharide comprising beta-1,6-linked glucosamine residues, the saccharide comprising at least five sugar residues, or binds to both.
33. The antibody or antigen-binding fragment thereof of any preceding or following embodiment/feature/aspect, wherein the antibody or antigen-binding fragment thereof binds to deacetylated beta-1,6-poly-N-acetyl glucosamine that is at least 90% deacetylated, or binds to a saccharide comprising beta-1,6-linked glucosamine residues, the saccharide comprising at least five sugar residues, or binds to both.
34. The antibody or antigen-binding fragment thereof of any preceding or following embodiment/feature/aspect, wherein the antibody or antigen-binding fragment thereof binds to deacetylated beta-1,6-poly-N-acetyl glucosamine that is fewer than about 60% N-acetylated, or binds to a saccharide comprising beta-1,6-linked glucosamine residues, the saccharide comprising at least five sugar residues, or binds to both.
35. The antibody or antigen-binding fragment thereof of any preceding or following embodiment/feature/aspect, wherein the antibody or antigen-binding fragment thereof binds to deacetylated beta-1,6-poly-N-acetyl glucosamine that is fewer than about 30% N-acetylated, or binds to a saccharide comprising beta-1,6-linked glucosamine residues, the saccharide comprising at least five sugar residues, or binds to both.
36. The antibody or antigen-binding fragment thereof of any preceding or following embodiment/feature/aspect, wherein the antibody or antigen-binding fragment thereof binds to deacetylated beta-1,6-poly-N-acetyl glucosamine that is fewer than about 15% N-acetylated, or binds to a saccharide comprising beta-1,6-linked glucosamine residues, the saccharide comprising at least five sugar residues, or binds to both.
37. The antibody or antigen-binding fragment thereof of any preceding or following embodiment/feature/aspect, wherein the antibody or antigen-binding fragment thereof binds to deacetylated beta-1,6-poly-N-acetyl glucosamine that is fewer than about 5% N-acetylated, or binds to a saccharide comprising beta-1,6-linked glucosamine residues, the saccharide comprising at least five sugar residues, or binds to both.
38. The antibody or antigen-binding fragment thereof of any preceding or following embodiment/feature/aspect, wherein the antibody or antigen-binding fragment thereof binds to deacetylated beta-1,6-poly-N-acetyl glucosamine is essentially free of N-acetylation.
39. The antibody or antigen-binding fragment thereof of any preceding or following embodiment/feature/aspect, wherein the antibody or antigen-binding fragment thereof binds to at least 50% deacetylated dPNAG with a dissociation constant (K_{D}) of less than about 5×10⁻⁸ M, less than about 1×10⁻⁸ M, less than about 5×10⁻⁹ M, less than about 1×10⁻⁹ M, less than about 5×10⁻¹⁰ M, less than about 1×10⁻¹⁰ M, less than about 5×10⁻¹¹ M, less than about 1×10⁻¹¹ M, less than about 5×10⁻¹² M, or less than about 1×10⁻¹² M, as measured by biolayer interferometry.
40. The antibody or antigen-binding fragment thereof of any preceding or following embodiment/feature/aspect, wherein the antibody competes with monoclonal antibody F598 in binding to dPNAG.
41. The antibody or antigen-binding fragment thereof of any preceding or following embodiment/feature/aspect, wherein the antibody or antigen-binding fragment thereof binds a biofilm.
42. The antibody or antigen-binding fragment thereof of any preceding or following embodiment/feature/aspect, wherein the biofilm comprises PNAG, or dPNAG, or both.
43. The antibody or antigen-binding fragment thereof of any preceding or following embodiment/feature/aspect, wherein the biofilm comprises one or more bacteria.
44. The antibody or antigen-binding fragment thereof of any preceding or following embodiment/feature/aspect, wherein the antibody or antigen-binding fragment thereof promotes opsonic killing of one or more bacteria.
45. The antibody or antigen-binding fragment thereof of any preceding or following embodiment/feature/aspect, wherein the heavy chain is a full-length heavy chain and the light chain is a full-length light chain.
46. The antibody or antigen-binding fragment thereof of any preceding or following embodiment/feature/aspect, wherein the antigen-binding fragment is a Fab, Fv, scFv, Fab′, or (Fab′)2, or any combination thereof.
47. The antibody or antigen-binding fragment thereof of any preceding or following embodiment/feature/aspect, wherein the antibody or antibody fragment comprises an IgG1 constant region.
48. The antibody or antigen-binding fragment thereof of any preceding or following embodiment/feature/aspect, wherein the antibody or antibody fragment is a monoclonal antibody.
49. The antibody or antigen-binding fragment thereof of any preceding or following embodiment/feature/aspect, wherein the antibody or antigen-binding fragment thereof is a chimeric antibody or antigen-binding fragment thereof.
50. The antibody or antigen-binding fragment thereof of any preceding or following embodiment/feature/aspect, wherein the antibody is a human antibody or a humanized antibody.
51. The antibody of any preceding or following embodiment/feature/aspect, wherein the antibody is a caninized antibody, a felinized antibody, or an equinized antibody, or any combination thereof.
52. The antibody of any preceding or following embodiment/feature/aspect, wherein the antibody is bispecific.
53. The antibody of any preceding or following embodiment/feature/aspect, wherein the bispecific antibody comprises two different specificities to dPNAG.
54. The antibody of any preceding or following embodiment/feature/aspect, wherein the bispecific antibody comprising specificity to dPNAG and a second antigen.
55. An antibody or antigen-binding fragment thereof of any preceding or following embodiment/feature/aspect conjugated to a detectable label.
56. The antibody or antigen-binding fragment thereof of any preceding or following embodiment/feature/aspect, wherein the label is a fluorescent label.
57. An antibody or antigen-binding fragment thereof of any preceding or following embodiment/feature/aspect conjugated to a therapeutic molecule
58. The antibody or antigen-binding fragment thereof of any preceding or following embodiment/feature/aspect, conjugated to an immunomodulator, a cytokine, a cytotoxic agent, a chemotherapeutic agent, a diagnostic agent, an antiviral agent, an antimicrobial agent, or a drug, or any combination thereof.
59. A pharmaceutical composition comprising the antibody or antigen-binding fragment thereof of any preceding or following embodiment/feature/aspect, and a pharmaceutically acceptable carrier.
60. A vaccine composition comprising an antibody or antigen-binding fragment thereof of any preceding or following embodiment/feature/aspect and beta-(1,6)-poly-N-acetylglucosamine.
61. A composition comprising two or more different antibodies or antigen-binding fragments thereof of any preceding or following embodiment/feature/aspect.
62. A composition comprising an antibody or antigen-binding fragment of any preceding or following embodiment/feature/aspect in combination with a second antibody or antigen-binding fragment thereof.
63. The antibody or antigen-binding fragment thereof of any preceding or following embodiment/feature/aspect, wherein the second antibody or antigen-binding fragment thereof binds to PNAG, or dPNAG, or both.
64. The antibody or antigen-binding fragment thereof of any preceding or following embodiment/feature/aspect, wherein the second antibody or antigen-binding fragment thereof binds to a polysaccharide.
65. The antibody or antigen-binding fragment thereof of any preceding or following embodiment/feature/aspect, wherein the second antibody or antigen-binding fragment thereof binds to a biofilm.
66. The second antibody or antigen-binding fragment thereof of any preceding or following embodiment/feature/aspect produced from a deposited hybridoma having Accession No. PTA-5931 (F598).
67. A composition comprising an antibody or antigen-binding fragment of any preceding or following embodiment/feature/aspect in combination with an antibiotic.
68. A kit comprising an antibody or antigen-binding fragment of any preceding or following embodiment/feature/aspect.
69. The kit of any preceding or following embodiment/feature/aspect, wherein the antibody or antigen-binding fragment is a primary antibody or primary antigen-binding fragment thereof, and the kit further comprises a secondary antibody or secondary antigen-binding fragment thereof that binds the primary antibody or primary antigen-binding fragment thereof.
70. The kit of any preceding or following embodiment/feature/aspect further comprising an antibiotic.
71. A substrate treated with an antibody or antigen-binding fragment thereof of any preceding or following embodiment/feature/aspect.
72. The substrate of any preceding or following embodiment/feature/aspect comprising a biological substrate, an artificial substrate, or both.
73. The substrate of any preceding or following embodiment/feature/aspect comprising a surgical implant, a dental implant, a nasal implant, an optical implant, or a dermally applied device, a prosthesis, or any combination thereof.
74. The surgical implant of any preceding or following embodiment/feature/aspect comprising a stent, a catheter, a cannula, an artificial joint, a prosthesis, or a pace-maker, or any combination thereof.
75. An isolated nucleic acid encoding the antibody or antigen-binding fragment thereof of any preceding or following embodiment/feature/aspect.
76. A host cell comprising the nucleic acid of any preceding or following embodiment/feature/aspect.
77. A method of producing an antibody or antigen-binding fragment thereof comprising culturing the host cell of any preceding or following embodiment/feature/aspect and isolating the antibody or antigen-binding fragment thereof.
78. A method of administering an effective amount of the antibody or antigen-binding fragment thereof of any preceding or following embodiment/feature/aspect to a patient in need thereof 79. The method of any preceding or following embodiment/feature/aspect, wherein the patient has or is at risk for a microbial infection.
80. The method of any preceding or following embodiment/feature/aspect, wherein the microbial infection is a bacterial infection.
81. The method of any preceding or following embodiment/feature/aspect, wherein the bacterial infection is characterized by one or more bacteria that produce poly-N-acetyl glucosamine (PNAG), or deacetylated poly-N-acetyl glucosamine (dPNAG), or both.
82. The method of any preceding or following embodiment/feature/aspect, wherein the microbial infection is characterized by the formation of a biofilm in a patient.
83. The method of any preceding or following embodiment/feature/aspect, wherein the biofilm is characterized by poly-N-acetyl glucosamine (PNAG), or deacetylated poly-N-acetyl glucosamine (dPNAG), or both.
84. The method of any preceding or following embodiment/feature/aspect, the antibody or antigen-binding fragment thereof is administered so as to provide passive immunity to the patient.
85. The method of any preceding or following embodiment/feature/aspect, wherein the patient has had, is undergoing, or is scheduled to undergo a surgical procedure.
86. The method of any preceding or following embodiment/feature/aspect, wherein the surgical procedure comprises implantation of a structure susceptible for formation of a biofilm.
87. The method of any preceding or following embodiment/feature/aspect, wherein the surgical procedure is the installation of a surgical implant in the patient.
88. The method of any preceding or following embodiment/feature/aspect, wherein the surgical implant is a stent, a catheter, a cannula, an artificial joint, a prosthesis, or a pace-maker, or any combination thereof.
89. The method of any preceding or following embodiment/feature/aspect, wherein the patient is a mammal.
90. The method of any preceding or following embodiment/feature/aspect, wherein the patient is human.
91. The method of any preceding or following embodiment/feature/aspect, wherein the microbial infection comprises a lung infection, a joint infection, an endocardial infection, a skin infection, a soft tissue infection, or septicemia, or any combination thereof.
92. The method of any preceding or following embodiment/feature/aspect, wherein the microbial infection comprises one or more bacteria of the genus Staphylococcus.
93. The method of any preceding or following embodiment/feature/aspect, wherein the one or more staphylococcal bacteria comprise S. epidermidis, or S. aureus, or both.
94. The method of any preceding or following embodiment/feature/aspect, wherein the microbial infection comprises one or more bacteria selected from E. coli, Yersinia pestis, Yersinia entercolitica, Xanthomonas axonopodis, Pseudomonas fluorescens, Actinobacillus actinomycetemcomitans, Actinobacillus pleuropneumoniae, Ralstonia solanacearum, Bordetella pertussis, Bordetella parapertussis, and Bordetella bronchiseptica.
95. The method of any preceding or following embodiment/feature/aspect, wherein the antibody or antigen-binding fragment thereof is administered at a dose of from about 0.1 mg/kg to about 100 mg/kg.
96. The method of any preceding or following embodiment/feature/aspect, wherein the administered dose achieves a serum level of anti-dPNAG antibody of from about 1.0 μg/mL to about 100 μg/ml.
97. The method of any preceding or following embodiment/feature/aspect, wherein the opsonic activity of the patient's serum remains essentially constant for from about 5 days to about 150 days after administration of the antibody or antigen binding fragment thereof.
98. The method of any preceding or following embodiment/feature/aspect, wherein the antibody or antigen-binding fragment thereof has a serum half-life of from about 1 day to about 100 days.
99. The method of any preceding or following embodiment/feature/aspect, wherein the antibody or antigen-binding fragment thereof is administered as a single dose.
100. The method of any preceding or following embodiment/feature/aspect, wherein the dosing and scheduling of at least one dose is based upon a determination of the serum half-life of the antibody or antibody binding fragment thereof in the patient.
101. The method of any preceding or following embodiment/feature/aspect, wherein dosing and scheduling of at least one dose is based upon a determination of the in vitro opsonic activity of the patient's serum against PNAG-expressing bacteria.
102. The method of any preceding or following embodiment/feature/aspect, further comprising determining an effective serum titer of the administered antibody using an in vitro opsonophagocytosis assay.
103. The method of any preceding or following embodiment/feature/aspect, wherein the antibody or antigen-binding fragment thereof is administered by intravenous infusion.
104. The method of any preceding or following embodiment/feature/aspect, wherein the antibody or antigen-binding fragment is administered systemically.
105. The method of any preceding or following embodiment/feature/aspect, wherein the antibody or antigen-binding fragment thereof is administered locally to the site of a wound, a body cavity, or an implanted medical device.
106. The method of any preceding or following embodiment/feature/aspect, wherein the antibody or antigen-binding fragment thereof is applied to a medical device before implantation, during implantation, or after implantation, or any combination thereof.
107. The method of any preceding or following embodiment/feature/aspect, wherein the antibody or an antigen-binding fragment thereof is administered in a controlled release formulation.
108. The method of any preceding or following embodiment/feature/aspect further comprising administering one or more polysaccharides.
109. The method of any preceding or following embodiment/feature/aspect, wherein the polysaccharide has an average molecular weight of from about 10 kDa to about 1,000 kDa.
110. The method of any preceding or following embodiment/feature/aspect further comprising administering deacetylated beta-1,6-poly-N-acetyl glucosamine (dPNAG).
111. The method of any preceding or following embodiment/feature/aspect further comprising administering at least 50% deacetylated beta-1,6-poly-N-acetyl glucosamine.
112. The method of any preceding or following embodiment/feature/aspect further comprising administering an adjuvant.
113. The method of any preceding or following embodiment/feature/aspect further comprising administering a second antibody or antigen binding fragment thereof.
114. The method of any preceding or following embodiment/feature/aspect wherein the second antibody or antigen-binding fragment thereof is produced from a deposited hybridoma having Accession No. PTA-5931 (F598).
115. The method of any preceding or following embodiment/feature/aspect further comprising administering one or more antibiotics.
116. A method of inhibiting formation of dPNAG-containing microbial biofilm on a substrate comprising contacting the substrate with an effective amount the antibody or antigen-binding fragment thereof of any preceding or following embodiment/feature/aspect.
117. A method of disrupting a biofilm, comprising contacting the biofilm with an effective amount of the antibody or antigen-binding fragment thereof of any preceding or following embodiment/feature/aspect.
118. A method of detecting a biofilm in a patient, comprising administering to the patient the antibody or antigen-binding fragment thereof of any preceding or following embodiment/feature/aspect, and detecting binding of the antibody or antigen-binding fragment to the biofilm.
119. A method of detecting a biofilm in a sample obtained from a patient comprising contacting the sample with the antibody or antigen-binding fragment thereof of any preceding or following embodiment/feature/aspect.
120. A method for detecting a biofilm on a substrate comprising contacting the substrate with the antibody or antigen-binding fragment thereof of any preceding or following embodiment/feature/aspect.

The present disclosure can include any combination of these various features or embodiments above and/or below as set forth in sentences and/or paragraphs. Any combination of disclosed features herein is considered part of the present disclosure. Applicants specifically incorporate the entire contents of all cited references in this disclosure. Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. The scope of the disclosure is not limited to the specific values recited when defining a range.

All references cited in this specification are herein incorporated by reference as though each reference was specifically and individually indicated to be incorporated by reference. The citation of any reference is for its disclosure prior to the filing date and should not be construed as an admission that the present disclosure is not entitled to claim any particular subject matter disclosed herein.

It will be understood that each of the elements described above, or two or more together may also find a useful application in other types of methods differing from the type described above. Without further analysis, the foregoing will so fully reveal the gist of the present disclosure that others can readily adapt it for various uses while still falling within the scope of the claimed subject matter. The foregoing embodiments are presented by way of example only.

1. An antibody or antigen-binding fragment thereof comprising:
a heavy chain variable region comprising:
a heavy chain complementarity determining region 1 (CDRH1) having an amino acid sequence comprising SEQ ID NO: 4 or SEQ ID NO: 32,
a heavy chain complementarity determining region 2 (CDRH2) having an amino acid sequence comprising SEQ ID NO: 6, and
a heavy chain complementarity determining region 3 (CDRH3) having an amino acid sequence of SEQ ID NO: 7 or SEQ ID NO: 35; and
a light chain variable region comprising:
a light chain complementarity determining region (CDRL1) having an amino acid sequence comprising SEQ ID NO: 13 or 25,
a light chain complementarity determining region (CDRL2) having an amino acid sequence comprising the amino acid sequence GAS, and
a light chain complementarity determining region (CDRL3) having an amino acid sequence comprising SEQ ID NO: 16 or 27,
wherein the antibody or antigen-binding fragment thereof binds to a saccharide comprising beta-1,6-linked glucosamine residues, the saccharide comprising at least five sugar residues,
wherein an amino acid sequence of the heavy chain variable region excludes SEQ. ID NO: 1, and
wherein an amino acid sequence of the light chain variable region excludes SEQ ID NO: 2.

2. The antibody or antigen-binding fragment thereof of claim 1, comprising:
a heavy chain variable region having a sequence of SEQ ID NO: 3, 31, or 34, and a light chain variable region having a sequence of SEQ ID NO: 12; or
a heavy chain variable region having a sequence of SEQ ID NO: 3, and a light chain variable region having a sequence of SEQ ID NO: 24.

3. The antibody or antigen-binding fragment thereof of claim 1, wherein:
the heavy chain variable region has an amino acid sequence comprising the CDRH1 sequence comprising SEQ ID NO: 4, the CDRH2 sequence comprising SEQ ID NO: 6, and the CDRH3 sequence comprising SEQ ID NO: 7;
the heavy chain variable region has an amino acid sequence comprising the CDRH1 sequence comprising SEQ ID NO: 32, the CDRH2 sequence comprising SEQ ID NO: 6, and the CDRH3 sequence comprising SEQ ID NO: 7; or
the heavy chain variable region has an amino acid sequence comprising the CDRH1 sequence comprising SEQ ID NO: 4, the CDRH2 sequence comprising SEQ ID NO: 6, and the CDRH3 sequence comprising SEQ ID NO: 35.

4. The antibody or antigen-binding fragment thereof of claim 1, wherein:
the heavy chain variable region has an amino acid sequence comprising SEQ ID NO: 3;
wherein the heavy chain variable region has an amino acid sequence comprising SEQ ID NO: 31; or
wherein the heavy chain variable region has an amino acid sequence comprising SEQ ID NO: 34.

5. The antibody or antigen-binding fragment thereof of claim 1, wherein:
the light chain variable region has an amino acid sequence comprising the CDRL1 sequence comprising SEQ ID NO: 13, the CDRL2 sequence comprising the amino acid sequence GAS, and the CDRL3 sequence comprising SEQ ID NO: 16; or
the light chain variable region has an amino acid sequence comprising the CDRL1 sequence comprising SEQ ID NO: 25, the CDRL2 sequence comprising the amino acid sequence GAS, and the CDRL3 sequence comprising SEQ ID NO: 27.

6. The antibody or antigen-binding fragment thereof of claim 1, wherein;
the light chain variable region has an amino acid sequence comprising SEQ ID NO: 12; or
the light chain variable region has an amino acid sequence comprising SEQ ID NO: 24.

7.-12. (canceled)

13. The antibody or antigen-binding fragment thereof of claim 1, wherein:
the heavy chain variable region has an amino acid sequence comprising SEQ ID NO: 3, and the light chain variable region has an amino acid sequence comprising SEQ ID NO: 12;
the heavy chain variable region has an amino acid sequence comprising SEQ ID NO: 3, and the light chain variable region has an amino acid sequence comprising SEQ ID NO: 24;
the heavy chain variable region has an amino acid sequence comprising SEQ ID NO: 31, and the light chain variable region has an amino acid sequence comprising SEQ ID NO: 12; or
the heavy chain variable region has an amino acid sequence comprising SEQ ID NO: 34, and the light chain variable region has an amino acid sequence comprising SEQ ID NO: 12.

14.-16. (canceled)

17. The antibody or antigen-binding fragment thereof of claim 1, wherein the saccharide comprises an oligosaccharide, or a polysaccharide, or both.

18. The antibody or antigen-binding fragment thereof of claim 1, wherein;
the saccharide comprises a population of polysaccharides differing in number of beta-1,6-linked glucosamine residues free of N-acetylation and number of beta-1,6-linked N-acetyl glucosamine residues; and/or
the saccharide comprises a population of polysaccharides differing in percent content of beta-1,6-linked glucosamine residues free of N-acetylation and percent content of beta-1,6-linked N-acetyl glucosamine residues.

19. (canceled)

20. The antibody or antigen-binding fragment thereof of claim 1, wherein the antibody or antigen-binding fragment thereof binds to:
a pentasaccharide comprising four beta-1,6-linked glucosamine residues free of N-acetylation, and one beta-1,6-linked N-acetyl glucosamine residue;
a pentasaccharide comprising three beta-1,6-linked glucosamine residues free of N-acetylation, and two beta-1,6-linked N-acetyl glucosamine residues;
a pentasaccharide comprising five beta-1,6-linked glucosamine residues free of N-acetylation;
a polysaccharide comprising beta-1,6-linked glucosamine, wherein the polysaccharide comprises fewer than 50% beta-1,6-linked-N-acetyl glucosamine residues;
a polysaccharide comprising beta-1,6-linked glucosamine, wherein the polysaccharide comprises fewer than 90% beta-1,6-linked-N-acetyl glucosamine residues; or
a polysaccharide comprising beta-1,6-linked glucosamine residues, wherein the polysaccharide comprises fewer than 50% beta-1,6-linked-N-acetyl glucosamine residues, with a dissociation constant (K_{D}) of less than about 5×10⁻⁸ M, less than about 1×10⁻⁸ M, less than about 5×10⁻⁹ M, less than about 1×10⁻⁹ M, less than about 5×10⁻¹⁰ M, less than about 1×10⁻¹⁰ M, less than about 5×10⁻¹¹ M, less than about 1×10⁻¹¹ M, less than about 5×10⁻¹² M, or less than about 1×10⁻¹² M, as measured by biolayer interferometry.

21. The antibody or antigen-binding fragment thereof of claim 20, wherein:
the beta-1,6-linked N-acetyl glucosamine residue is a terminal residue; or
the pentasaccharide comprises two adjacent beta-1,6-linked glucosamine residues free of N-acetylation.

22.-27. (canceled)

28. The antibody or antigen-binding fragment thereof of claim 1, wherein the antibody or antigen-binding fragment thereof binds a biofilm, wherein the biofilm comprises beta-1,6-linked glucosamine residues and beta-1,6-linked-N-acetyl glucosamine residues

29. The antibody or antigen-binding fragment thereof of claim 1, wherein the antibody or antigen-binding fragment thereof promotes opsonic killing of one or more bacteria.

30. The antibody or antigen-binding fragment thereof of claim 1, wherein:
the heavy chain is a full-length heavy chain and the light chain is a full-length light chain;
the antigen-binding fragment is a Fab, Fv, scFv, Fab′, or (Fab′)2, or any combination thereof; or
the antibody or antibody fragment comprises an IgG1 constant region.

31.-32. (canceled)

33. The antibody or antigen-binding fragment thereof of claim 1, wherein:
the antibody or antibody fragment is a monoclonal antibody;
the antibody or antigen-binding fragment thereof is a chimeric antibody or antigen-binding fragment thereof; or
the antibody is a human antibody or a humanized antibody.

34.-35. (canceled)

36. The antibody of claim 1, wherein the antibody is bispecific.

37. The bispecific antibody of claim 36, wherein the bispecific antibody can bind to beta-1,6-linked glucosamine residues and beta-1,6-linked-N-acetyl glucosamine residues.

38. (canceled)

39. The antibody or antigen-binding fragment thereof of claim 1, conjugated to a detectable label, a therapeutic molecule, an immunomodulator, a cytokine, a cytotoxic agent, a chemotherapeutic agent, a diagnostic agent, an antiviral agent, an antimicrobial agent, or a drug, or any combination thereof.

40. A composition comprising the antibody or antigen-binding fragment thereof of claim 1, and a pharmaceutically acceptable carrier.

41. A composition comprising,
two or more different antibodies or antigen-binding fragments thereof of claim 1; and/or
an antibody or antigen-binding fragment of claim 1 in combination with a second antibody or antigen-binding fragment thereof.

42. (canceled)

43. The composition of claim 41, wherein the second antibody or antigen-binding fragment thereof:
binds to beta-1,6-polyglucosamine, beta-1,6-poly-N-acetyl glucosamine (PNAG), partially deacetylated PNAG (dPNAG), or any combination thereof;
binds to a pentasaccharide comprising five beta-1,6-linked N-acetyl glucosamine residues; and/or
is produced from a deposited hybridoma having Accession No. PTA-5931 (F598).

44.-45. (canceled)

46. A substrate treated with an antibody or antigen-binding fragment thereof of claim 1.

47. An isolated nucleic acid encoding the antibody or antigen-binding fragment thereof of claim 1.

48. A host cell comprising the nucleic acid of claim 47.

49. A method of producing an antibody or antigen-binding fragment thereof comprising culturing the host cell of claim 48 and isolating the antibody or antigen-binding fragment thereof.

50. A method, comprising of administering an effective amount of the antibody or antigen-binding fragment thereof of claim 1 to a patient in need thereof

51. The method of claim 50, wherein the patient has or is at risk for a microbial infection.

52. The method of claim 51, wherein the microbial infection is a bacterial infection.

53. The method of claim 52, wherein the bacterial infection is characterized by one or more bacteria that produce poly-N-acetyl glucosamine (PNAG), or partially deacetylated poly-N-acetyl glucosamine (dPNAG), or both.

54. The method of claim 50, wherein:
opsonic activity of the patient's serum remains essentially constant for from about 5 days to about 150 days after administration of the antibody or antigen binding fragment thereof or composition comprising the same; and/or
further comprising administering a second antibody or antigen binding fragment thereof or composition comprising the same.

55. (canceled)

56. The method of claim 54, wherein:
the second antibody or antigen-binding fragment thereof is produced from a deposited hybridoma having Accession No. PTA-5931 (F598); and/or
the antibody or antigen-binding fragment thereof and the second antibody or antigen-binding fragment thereof are administered as an admixture.

57. (canceled)

58. A method, comprising:
inhibiting formation of a microbial biofilm on a substrate comprising contacting the substrate with an effective amount of the antibody or antigen-binding fragment thereof of claim 1, wherein the microbial biofilm comprises beta-1,6-linked glucosamine residues and beta-1,6-linked-N-acetyl glucosamine residues;
disrupting a biofilm comprising contacting the biofilm with an effective amount of the antibody or antigen-binding fragment thereof of claim 1, wherein the microbial biofilm comprises beta-1,6-linked glucosamine residues and beta-1,6-linked-N-acetyl glucosamine residues; and/or
detecting a biofilm on a substrate comprising contacting the substrate with the antibody or antigen-binding fragment thereof of claim 1, wherein the biofilm comprises beta-1,6-linked glucosamine residues and beta-1,6-linked-N-acetyl glucosamine residues.

59.-60. (canceled)


SEQUENCE LISTING (57 sequences)

SEQ ID NO: 1 - protein, 118 aa, Homo sapiens
EVQLLESGGGLVQPGGSLRLSCAVSGFPFSSYWMSWVRQAPGKGLEWVANIKEDGSEKYYVDSVKGRFTISRDNAKNSLYLQMNSLRAEDTAVYYCATDPDVGLSDSWGQGTLVTVSS

SEQ ID NO: 2 - protein, 108 aa, Homo sapiens
ETTLTQSPATLSLSPGDRATHSCRASQSVSRSYLAWYQQKPGQTPRLFIYGASNRATGIPDRFSGSGSGTYFTLTISRLEPEDFAVYYCQQYDSAPDTFGQGTRLEIK

SEQ ID NO: 3 - protein, 118 aa, synthetic construct
  REGION 1..118: note = TG10 Heavy Chain Variable Region
EVQLVESGGGLVQPGGSLRLSCAVSGFPFSSYWMSWVRQAPGKGLEWVANIKEDGSEKYYVDSVKGRFTISRDNAKNSLYLQMNSLRAEDTAVYYCATDPDVGLSDSWGQGTLVTVSS

SEQ ID NO: 4 - protein, 8 aa, synthetic construct
  REGION 1..8: note = Fragment of TG10 HC Variable Region
GFPFSSYW

SEQ ID NO: 5 - protein, 12 aa, synthetic construct
  REGION 1..12: note = Fragment of TG10 HC Variable Region
VSGFPFSSYWMS

SEQ ID NO: 6 - protein, 8 aa, synthetic construct
  REGION 1..8: note = Fragment of TG10 HC Variable Region
IKEDGSEK

SEQ ID NO: 7 - protein, 11 aa, synthetic construct
  REGION 1..11: note = Fragment of TG10 HC Variable Region
ATDPDVGLSDS

SEQ ID NO: 8 - protein, 25 aa, synthetic construct [sequence omitted]

SEQ ID NO: 9 - protein, 17 aa, synthetic construct
  REGION 1..17: note = Fragment of TG10 HC Variable Region
MSWVRQAPGKGLEWVAN

SEQ ID NO: 10 - protein, 38 aa, synthetic construct
  REGION 1..38: note = Fragment of TG10 HC Variable Region
YYVDSVKGRFTISRDNAKNSLYLQMNSLRAEDTAVYYC

SEQ ID NO: 11 - protein, 11 aa, synthetic construct
  REGION 1..11: note = Fragment of TG10 HC Variable Region
WGQGTLVTVSS

SEQ ID NO: 12 - protein, 108 aa, synthetic construct
  REGION 1..108: note = TG10 Light Chain Variable Region
EIVLTQSPGTLSLSPGDRATHSCRASQSVSRSYLAWYQQKPGQTPRLFIYGASNRATGIPDRFSGSGSGTYFTLTISRLEPEDFAVYYCQQYDSAPDTFGQGTRLEIK

SEQ ID NO: 13 - protein, 7 aa, synthetic construct
  REGION 1..7: note = Fragment of TG10 LC Variable Region
QSVSRSY

SEQ ID NO: 14 - protein, 19 aa, synthetic construct
  REGION 1..19: note = Fragment of TG10 LC Variable Region
HSCRASQSVSRSYLAWYQQ

SEQ ID NO: 15 - protein, 17 aa, synthetic construct
  REGION 1..17: note = Fragment of TG10 LC Variable Region
TPRLFIYGASNRATGIP

SEQ ID NO: 16 - protein, 9 aa, synthetic construct
  REGION 1..9: note = Fragment of TG10 LC Variable Region
QQYDSAPDT

SEQ ID NO: 17 - protein, 13 aa, synthetic construct
  REGION 1..13: note = Fragment of TG10 LC Variable Region
YCQQYDSAPDTFG

SEQ ID NO: 18 - protein, 26 aa, synthetic construct
  REGION 1..26: note = Fragment of TG10 LC Variable Region
EIVLTQSPGTLSLSPGDRATHSCRAS

SEQ ID NO: 19 - protein, 17 aa, synthetic construct
  REGION 1..17: note = Fragment of TG10 LC Variable Region
LAWYQQKPGQTPRLFIY

SEQ ID NO: 20 - protein, 36 aa, synthetic construct
  REGION 1..36: note = Fragment of TG10 LC Variable Region
NRATGIPDRFSGSGSGTYFTLTISRLEPEDFAVYYC

SEQ ID NO: 21 - protein, 10 aa, synthetic construct
  REGION 1..10: note = Fragment of TG10 LC Variable Region
FGQGTRLEIK

SEQ ID NO: 22 - protein, 118 aa, Homo sapiens
EVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCARDPDVGLSDYWGQGTLVTVSS

SEQ ID NO: 23 - protein, 108 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPDTFGQGTRLEIK

SEQ ID NO: 24 - protein, 108 aa, unidentified
  REGION 1..108: note = Based on antibody sequence.
EIVLTQSPGTLSLSPGERATLSCRASQSVSSSYLAWYQQKPGQTPRLLIYGASSRATGIPDRFSGSGSGTDFTLTISRLEPEDFAVYYCQQYDSSPDTFGQGTRLEIK

SEQ ID NO: 25 - protein, 7 aa, unidentified
  REGION 1..7: note = Fragment of 266 LC Variable Region
QSVSSSY

SEQ ID NO: 26 - protein, 29 aa, unidentified
  REGION 1..29: note = Fragment of 266 LC Variable Region
GERATLSCRASQSVSSSYLAWYQQKPGQT

SEQ ID NO: 27 - protein, 9 aa, unidentified
  REGION 1..9: note = Fragment of 266 LC Variable Region.
QQYDSSPDT

SEQ ID NO: 28 - protein, 26 aa, unidentified [sequence omitted]

SEQ ID NO: 29 - protein, 17 aa, unidentified
  REGION 1..17: note = Fragment of 266 LC Variable Region
LAWYQQKPGQTPRLLIY

SEQ ID NO: 30 - protein, 36 aa, unidentified [sequence omitted]

SEQ ID NO: 31 - protein, 118 aa, synthetic construct
  REGION 1..118: note = Site directed mutation of TG10 HC Variable Region
EVQLVESGGGLVQPGGSLRLSCAVSGFPFSSAWMSWVRQAPGKGLEWVANIKEDGSEKYYVDSVKGRFTISRDNAKNSLYLQMNSLRAEDTAVYYCATDPDVGLSDSWGQGTLVTVSS

SEQ ID NO: 32 - protein, 8 aa, synthetic construct
  REGION 1..8: note = Fragment of TG10 HC Y37A HC Variable Region
GFPFSSAW

SEQ ID NO: 33 - protein, 12 aa, synthetic construct
  REGION 1..12: note = Fragment of TG10 HC Y37A HC Variable Region
VSGFPFSSAWMS

SEQ ID NO: 34 - protein, 118 aa, synthetic construct
  REGION 1..118: note = Site directed mutation of TG10 HC Variable Region
EVQLVESGGGLVQPGGSLRLSCAVSGFPFSSYWMSWVRQAPGKGLEWVANIKEDGSEKYYVDSVKGRFTISRDNAKNSLYLQMNSLRAEDTAVYYCATDPDVGLSASWGQGTLVTVSS

SEQ ID NO: 35 - protein, 11 aa, synthetic construct
  REGION 1..11: note = Fragment of TG10 D116A HC Variable Range
ATDPDVGLSAS

SEQ ID NO: 36 - protein, 118 aa, synthetic construct
  REGION 1..118: note = Site directed mutation of TG10 HC Variable Region
EVQLVESGGGLVQPGGSLRLSCAVSGFPFSSYWMSWVRQAPGKGLEWVANIKEDGSEKYYVDSVKGRFTISRDNAKNSLYLQMNSLRAEDTAVYYCATDADVGLSDSWGQGTLVTVSS

SEQ ID NO: 37 - protein, 118 aa, synthetic construct
  REGION 1..118: note = Site directed mutation of TG10 HC Variable Region
EVQLVESGGGLVQPGGSLRLSCAVSGFPFSSYWMSWVRQAPGKGLEWVANIKADGSEKYYVDSVKGRFTISRDNAKNSLYLQMNSLRAEDTAVYYCATDPDVGLSDSWGQGTLVTVSS

SEQ ID NO: 38 - protein, 118 aa, synthetic construct
  REGION 1..118: note = Site directed mutation of TG10 HC Variable Region
EVQLVESGGGLVQPGGSLRLSCAVSGFPFSSYWMSWVRQAPGKGLEWVANIKEDGSEKAYVDSVKGRFTISRDNAKNSLYLQMNSLRAEDTAVYYCATDPDVGLSDSWGQGTLVTVSS

SEQ ID NO: 39 - protein, 118 aa, synthetic construct
  REGION 1..118: note = Site directed mutation of TG10 HC Variable Region
EVQLVESGGGLVQPGGSLRLSCAVSGFPFSSYWMSWVRQAPGKGLEWVANIKEDGSEKYYVDSVKGRFTISRDNAKNSLYLQMNSLRAEDTAVYYCATDPDRGLSDSWGQGTLVTVSS

SEQ ID NO: 40 - protein, 108 aa, synthetic construct
  REGION 1..108: note = Site directed mutation of TG10 LC Variable Region
EIVLTQSPGTLSLSPGDRATHSCRASQSVSRSYLAWYQQKPGQTPRLFIAGASNRATGIPDRFSGSGSGTYFTLTISRLEPEDFAVYYCQQYDSAPDTFGQGTRLEIK

SEQ ID NO: 41 - protein, 118 aa, synthetic construct
  REGION 1..118: note = Site directed mutation of TG10 HC Variable Region
EVQLVESGGGLVQPGGSLRLSCAVSGFPFSSYWMSWVRQAPGKGLEWVANIKEDGSEKYYVDSVKGRFTISRDNAKNSLYLQMNSLRAEDTAVYYCATAPDVGLSDSWGQGTLVTVSS

SEQ ID NO: 42 - protein, 118 aa, synthetic construct
  REGION 1..118: note = Site directed mutatation of TG10 HC Variable Region
EVQLVESGGGLVQPGGSLRLSCAVSGFPFSSYWMSWVRQAPGKGLEWVANIKEDGSEKYYVDSVKGRFTISRDNAKNSLYLQMNSLRAEDTAVYYCATDPAVGLSDSWGQGTLVTVSS

SEQ ID NO: 43 - protein, 110 aa, synthetic construct
  REGION 1..110: note = Site directed mutation of TG10 LC Variable Region
EIVLTQSPGTLSLSPGDRATHSCRASQSVSRSALAWYQQKPGQTPRLFIYGASNRATGIPDRFSGSGSGTYFTLTISRLEPEDFAVYYCQQYDSAPDTFGQGTRLEIKLC

SEQ ID NO: 44 - protein, 118 aa, unidentified
  REGION 1..118: note = Based on an antibody sequence.
EVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCARDPDGRIFDYWGQGTLVTVSS

SEQ ID NO: 45 - protein, 118 aa, unidentified
  REGION 1..118: note = Based on an antibody sequence.
QVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCARDPEGGLPDYWGQGTLVTVSS

SEQ ID NO: 46 - protein, 118 aa, unidentified
  REGION 1..118: note = Based on an antibody sequence.
EVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCARDPYMGLIDYWGQGTLVTVSS

SEQ ID NO: 47 - protein, 118 aa, synthetic construct
  REGION 1..118: note = Site directed mutatation of TG10 HC Variable Region
EVQLVESGGGLVQPGGSLRLSCAVSGFPFSSYAMSWVRQAPGKGLEWVANIKEDGSEKYYVDSVKGRFTISRDNAKNSLYLQMNSLRAEDTAVYYCATDPDVGLSDSWGQGTLVTVSS

SEQ ID NO: 48 - protein, 118 aa, synthetic construct
  REGION 1..118: note = Site directed mutatation of TG10 HC Variable Region
EVQLVESGGGLVQPGGSLRLSCAVSGFPFSSYWMSWVRQAPGKGLEWVANIKEDGSEKYYVDSVKGRFTISRDNAKNSLYLQMNSLRAEDTAVYYCATDPKVGLSDSWGQGTLVTVSS

SEQ ID NO: 49 - protein, 108 aa, synthetic construct
  REGION 1..108: note = Site directed mutatation of TG10 HC Variable Region
EIVLTQSPGTLSLSPGDRATHSCRASQSVSRSYLAWYQQKPGQTPRLFIYGASNRATGIPDRFSGSGSGTYFTLTISRLEPEDFAVYYCQQYDSAPATFGQGTRLEIK

SEQ ID NO: 50 - protein, 19 aa, Homo sapiens
MGWSCIILFLVATATGVHS

SEQ ID NO: 51 - DNA, 57 bp, Homo sapiens
  source 1..57: mol_type = other DNA
atgggctggagctgcatcattctgtttctggtggccacagccaccggcgtgcacagc

SEQ ID NO: 52 - DNA, 1404 bp, synthetic construct
  misc_feature 1..1404: note = Codon optimized sequence.
  source 1..1404: mol_type = other DNA
atgggctggtcctgcatcatcctgttcctggtggcaaccgcaacaggagtgcacagcgaggtgcagctgctggagtccggaggaggactggtgcagccaggaggaagcctgaggctgtcctgtgccgtgtctggcttcccctttagctcctactggatgtcttgggtgcgccaggcacctggcaagggactggagtgggtggccaacatcaaggaggacggctctgagaagtactatgtggatagcgtgaagggccggtttaccatctccagagacaacgccaagaacagcctgtatctgcagatgaatagcctgagggcagaggacaccgccgtgtactattgcgcaacagacccagatgtgggactgtccgattcttggggccagggcaccctggtgacagtgtctagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 53 - DNA, 705 bp, synthetic construct
  misc_feature 1..705: note = Codon optimized sequence.
  source 1..705: mol_type = other DNA
atgggctggtcctgcatcatcctgttcctggtggcaaccgcaacaggcgtgcacagcgagaccacactgacccagtccccagccacactgagcctgtccccaggcgaccgggccacacactcctgtagagcctctcagagcgtgtcccggtcttacctggcctggtatcagcagaagcccggccagacccctagactgtttatctacggcgcctctaacagggcaacaggcatcccagaccgcttcagcggatccggatctggcacctacttcaccctgacaatctctaggctggagcctgaggatttcgccgtgtactattgccagcagtacgacagcgccccagatacctttggccagggcacaagactggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 54 - DNA, 1404 bp, synthetic construct
  misc_feature 1..1404: note = Codon optimized sequence.
  source 1..1404: mol_type = other DNA
atgggctggtcctgcatcatcctgttcctggtggcaaccgcaacaggagtgcacagcgaggtgcagctggtggagtccggaggaggactggtgcagccaggaggaagcctgaggctgtcctgtgccgtgtctggcttcccctttagctcctactggatgtcttgggtgcgccaggcacctggcaagggactggagtgggtggccaacatcaaggaggacggctctgagaagtactatgtggatagcgtgaagggccggtttaccatctccagagacaacgccaagaacagcctgtatctgcagatgaatagcctgagggcagaggacaccgccgtgtactattgcgcaacagacccagatgtgggactgtccgattcttggggccagggcaccctggtgacagtgtctagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 55 - DNA, 705 bp, synthetic construct
  misc_feature 1..705: note = Codon optimized sequence.
  source 1..705: mol_type = other DNA
atgggctggtcctgcatcatcctgttcctggtggcaaccgcaacaggcgtgcacagcgagatcgtgctgacccagtccccaggcacactgagcctgtccccaggcgaccgggccacacactcctgtagagcctctcagagcgtgtcccggtcttacctggcctggtatcagcagaagcccggccagacccctagactgtttatctacggcgcctctaacagggcaacaggcatcccagaccgcttcagcggatccggatctggcacctacttcaccctgacaatctctaggctggagcctgaggatttcgccgtgtactattgccagcagtacgacagcgccccagatacctttggccagggcacaagactggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 56 - DNA, 477 bp, unidentified
  misc_feature 1..477: note = Coding sequence of antibody based sequence.
  source 1..477: mol_type = other DNA
tgctaagtaagcagtggtatcaacgcagagtacatgggggaagacaatgggtttgatttagattacatgggtgacttttctgttttatttccaatctcagataccaccggagaaattgtgttgacgcagtctccaggcaccctgtctttgtctccaggggaaagagccaccctctcctgcagggccagtcagagtgttagcagcagctacttagcctggtaccagcagaaacctggccagactcccaggctcctcatctatggtgcatccagcagggccactggcatcccagacaggttcagtggcagtgggtctgggacagacttcactctcaccatcagcagactggagcctgaagattttgcagtgtattactgtcagcagtatgatagctcacctgacaccttcggccaagggacacgactggagattaaacgaactgtggctgcaccatctgtcttcatcttcctggatctg

SEQ ID NO: 57 - protein, 122 aa, unidentified
  REGION 1..122: note = Antibody based sequence.
EIVLTQSPGTLSLSPGERATLSCRASQSVSSSYLAWYQQKPGQTPRLLIYGASSRATGIPDRFSGSGSGTDFTLTISRLEPEDFAVYYCQQYDSSPDTFGQGTRLEIKRTVAAPSVFIFLDL